SEQUENCE LISTING (10 sequences)

SEQ ID NO: 1 - protein, 5 aa, Homo sapiens
DSAMS

SEQ ID NO: 2 - protein, 19 aa, Homo sapiens
FIKSKTYGGTKEYAASVKG

SEQ ID NO: 3 - protein, 16 aa, Homo sapiens
GAPYGGNSDYYYGLDV

SEQ ID NO: 4 - protein, 11 aa, Homo sapiens
RTSQDVRGALA

SEQ ID NO: 5 - protein, 7 aa, Homo sapiens
DASSLET

SEQ ID NO: 6 - protein, 9 aa, Homo sapiens
QQFLDFPFT

SEQ ID NO: 7 - protein, 124 aa, synthetic construct
  REGION 1..124: note = RB-1 heavy chain with amino acid substitution
EVQLVESGGGLVRPGRSLRLSCTVSGFSFDDSAMSWVRQAPGKGLEWISFIKSKTYGGTKEYAASVKGRFTISRDDSKNIAYLQMNSLKTEDTAVYYCTRGAPYGGNSDYYYGLDVWGQGTTVT

SEQ ID NO: 8 - protein, 106 aa, Homo sapiens
MTQSPSSLSASVGDRVTITCRTSQDVRGALAWYQQKPGKAPKLLIFDASSLETGVPSRFSGSGSGTVFTLTISSLQPEDFAAYYCQQFLDFPFTFGQGTRLEIKRT

SEQ ID NO: 9 - protein, 457 aa, synthetic construct
  REGION 1..457: note = RB1+YTE heavy chain
EVQLVESGGGLVRPGRSLRLSCTVSGFSFDDSAMSWVRQAPGKGLEWISFIKSKTYGGTKEYAASVKGRFTISRDDSKNIAYLQMNSLKTEDTAVYYCTRGAPYGGNSDYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = RB1+YTE light chain
DIQMTQSPSSLSASVGDRVTITCRTSQDVRGALAWYQQKPGKAPKLLIFDASSLETGVPSRFSGSGSGTVFTLTISSLQPEDFAAYYCQQFLDFPFTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0217798 A1
(43) Pub. Date: Jul. 30, 2026

(54) FORMULATIONS OF ANTI-RSV ANTIBODIES AND METHODS OF USE THEREOF

(71) Applicant: Merck Sharp & Dohme LLC, Rahway, NJ (US)

(72) Inventors: Venus Hashemi, Manalapan, NJ (US); Arnab De, Burlington, MA (US); Chakravarthy Nachu Narasimhan, Scotch Plains, NJ (US)

(73) Assignee: Merck Sharp & Dohme LLC, Rahway, NJ (US)

(21) Appl. No.: 19/462,004

(22) Filed: Jan. 28, 2026

Related U.S. Application Data
(62) Division of application No. 17/285,156, filed on Oct. 14, 2019, now Pat. No. 12,565,526.
(60) Provisional application No. 62/747,418, filed on Oct. 18, 2018.

(51) Int. Cl. C07K 16/11; A61K 39/00; A61K 47/12; A61K 47/18

(52) U.S. Cl. CPC C07K 16/11; A61K 47/12; A61K 47/183; A61K 2039/54; A61K 2039/545; C07K 2317/565; C07K 2317/94

(57) ABSTRACT

The present invention relates to stable formulations comprising antibodies or antigen-binding fragments thereof that bind to respiratory syncytial virus (RSV). Also provided are methods of preventing and/or treating RSV-related diseases with the formulations of the invention.

### CROSS-REFERENCE TO RELATED APPLICATIONS

This application is a division of U.S. patent application Ser. No. 17/285,156, filed Apr. 14, 2021, which is a National Stage Entry under 35 U.S.C. 371 of PCT/US2019/056027, filed Oct. 14, 2019, which claims the benefit of U.S. Provisional Patent Application Ser. No. 62/747,418 filed Oct. 18, 2018, the entire contents of which are incorporated by reference herein.

### FIELD OF THE INVENTION

The invention relates to stable formulations comprising antibodies or antigen-binding fragments thereof that bind to respiratory syncytial virus (RSV). Also provided are methods of preventing and/or treating RSV-related diseases with the formulations of the invention.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jan. 6, 2026, is named 24641USDIVSEQ.xml and is 11,490 bytes in size.

### BACKGROUND OF THE INVENTION

Paramyxoviruses are enveloped negative-strand RNA viruses that are significant human and animal pathogens. Human Respiratory Syncytial Virus (hRSV, RSV) belongs to the family Paramyxoviridae, subfamily Pneumovirinae. Two subtypes, type A and type B, have been identified and are a major cause of severe and sometimes even fatal respiratory infections in children less than 6 months of age. Adults with underlying diseases, such as COPD, asthma, and cancer, and adults with immunocompromised status, including HIV or post transplantation, are also at risk of developing severe RSV infection. Fifteen percent of annual hospitalizations in adults over 50 years due to acute respiratory infection are caused by RSV. In the United States, RSV causes more than 100,000 hospitalizations annually, and it is estimated to cause about 160,000 deaths globally each year. Currently there is no vaccine for RSV, and a trial with a formalin-inactivated virus was associated with increased disease severity in infants upon infection with RSV. Other family members including Human Metapneumo Virus (hMPV) and Human Parainfluenza Virus (hPIV) are also responsible for acute respiratory illness similar to hRSV.

The hRSV genome is a single-stranded negative-sense RNA molecule of approximately 15 kb that encodes 11 proteins. Two of these proteins are the main surface glycoproteins of the virion. These are (i) the attachment (G) protein, which mediates virus binding to cells, and (ii) the fusion (F) protein, which promotes both fusion of the viral and cell membranes at the initial stages of the infectious cycle and fusion of the membrane of infected cells with those of adjacent cells to form characteristic syncytia. The attachment protein G binds cellular surface receptors and interacts with the F protein. This interaction triggers a conformational change in the F protein to induce membrane fusion, thereby releasing the viral ribonucleoprotein complex into the host cell cytoplasm.

Monoclonal antibodies against the F protein or the G protein have been shown to have neutralizing effects in vitro and prophylactic effects in vivo. See, e.g., Beeler and Coelingh 1989, J. Virol. 63:2941-50; Garcia-Barreno et al., 1989, J. Virol. 63:925-32; Taylor et al., 1984, Immunology 52: 137-142; Walsh et al., 1984, Infection and Immunity 43:756-758; and U.S. Pat. Nos. 5,842,307 and 6,818,216. Other hRSV antibodies are described in International Patent Application Nos. WO94/06448 and WO92/04381 and U.S. Pat. Nos. 8,221,759 and 9,963,500.

Antibodies for use in human subjects must be stored prior to use and transported to the point of administration. Reproducibly attaining a desired level of antibody drug in a subject requires that the drug be stored in a formulation that maintains the bioactivity of the drug. The need exists for stable formulations of anti-RSV antibodies for pharmaceutical use, e.g., for preventing and/or treating RSV-related diseases. Preferably, such formulations will exhibit a long shelf-life, be stable when stored and transported, and will be amenable to administration at high concentrations, e.g. for use in subcutaneous administration, as well as low concentrations, e.g. for intravenous administration.

The formulations of the invention are useful for subcutaneous delivery to a patient in need thereof. In order to deliver maximum therapeutic benefits to patients, it is desirable that formulations for subcutaneous (SC) delivery comprise a high antibody concentration (75-200 mg/ml). A high concentration of API is often required for SC formulations due to the historical bioavailability of 50-60% for SC injections and the expected dose range of an antibody product. However, high concentration of antibody, or antigen-binding fragment thereof, may contribute to other properties of the product which would be undesirable, e.g. low injectability due to increased viscosity and higher than physiological osmolality and increased aggregation. Therefore, it is preferred that an antibody product intended for SC administration balances the effects of concentration while maintaining a level of drug that will provide the highest therapeutic benefit. An ideal product comprises a high protein concentration, low viscosity, an osmolality similar to physiological conditions, and a low level of aggregation under typical storage conditions. Increased viscosity at high protein concentration may not only make it difficult to extract the product from its container with a syringe, but also to inject the necessary dose into a patient from the syringe (syringeability). Advantageously, embodiments of the invention provide formulations that comprise a high concentration of antibody, or antigen-binding fragment thereof, and a viscosity level that is acceptable for subcutaneous delivery. Additionally, the formulations of the invention do not lead to high levels of aggregation, as shown in more detail throughout the Examples.

### SUMMARY OF THE INVENTION

The present disclosure provides an anti-RSV antibody formulation, comprising: a) about 50 mg/mL to about 250 mg/mL of an anti-RSV antibody, or antigen-binding fragment thereof, b) about 5 mM to about 20 mM buffer; c) a stabilizer selected from the group consisting of: (i) about 6% to about 8% weight/volume (w/v) non-reducing sugar; (ii) about 25 mM to about 75 mM each of L-arginine, or a pharmaceutically acceptable salt of L-arginine, L-proline, or a pharmaceutically acceptable salt of L-proline; L-lysine, or a pharmaceutically acceptable salt of L-lysine; L-glutamate, or a pharmaceutically acceptable salt of L-glutamate, or a mixture of the amino acids described herein, and (iii) about 25 μM to 75 μM of a chelator; d) about 0.01% to about 0.10% non-ionic surfactant; and e) about 1 mM to about 20 mM anti-oxidant.

In another embodiment, the disclosure provides an anti-RSV antibody formulation, comprising: a) about 50 mg/mL to about 150 mg/mL of an anti-RSV antibody, or antigen-binding fragment thereof, b) about 5 mM to about 20 mM histidine; c) about 6% to about 8% weight/volume (w/v) sucrose; d) an excipient selected from about 25 mM to about 75 mM of L-arginine or a pharmaceutically acceptable salt of L-arginine, about 25 mM to about 75 mM L-lysine or a pharmaceutically acceptable salt of L-lysine, a mixture of about 25 mM to about 75 mM L-arginine and about 25 to about 75 mM L-glutamate, and a mixture of about 25 mM to about 75 mM of L-arginine(HCl) and about 25 to about 75 mM L-lysine(HCl); e) about 25 μM to 75 μM of DTPA; f) about 0.01% to about 0.10% polysorbate 80; and optionally g) about 1 mM to about 20 mM L-methionine.

In another embodiment, the disclosure provides an anti-RSV antibody formulation, comprising: a) about 100 mg/mL of an anti-RSV antibody, or antigen-binding fragment thereof, b) about 10 mM histidine; c) about 7% weight/volume (w/v) sucrose; d) an excipient selected from about 50 mM L-arginine(HCl), about 50 mM L-lysine(HCl), a mixture of about 25 mM L-arginine and about 25 mM L-glutamate, and a mixture of about 25 mM L-arginine(HCl) and about 25 mM L-lysine(HCl); e) about 50 μM DTPA; f) about 0.02% polysorbate 80; and optionally g) about 1 mM to 20 mM L-methionine.

In another embodiment, the disclosure provides an anti-RSV antibody formulation, comprising: a) 100 mg/mL of an anti-RSV antibody, or antigen-binding fragment thereof, b) 10 mM histidine; c) 7% weight/volume (w/v) sucrose; d) an excipient selected from 50 mM L-arginine(HCl), 50 mM L-lysine(HCl), a mixture of 25 mM L-arginine and 25 mM L-glutamate, and a mixture of 25 mM L-arginine(HCl) and 25 mM L-lysine(HCl); e) 50 μM DTPA; f) 0.02% polysorbate 80; and optionally g) 1 mM to 20 mM L-methionine.

In another embodiment, the disclosure provides an anti-RSV antibody formulation, comprising: a) 100 mg/mL of an anti-RSV antibody, or antigen-binding fragment thereof, b) 10 mM histidine; c) 7% weight/volume (w/v) sucrose; d) a mixture of 25 mM L-arginine(HCl) and 25 mM L-lysine(HCl); e) 50 μM DTPA; and f) 0.02% polysorbate 80.

In another embodiment, the disclosure provides an anti-RSV antibody formulation consisting of: a) 100 mg/mL of an anti-RSV antibody, or antigen-binding fragment thereof, b) 10 mM histidine; c) 7% weight/volume (w/v) sucrose; d) a mixture of 25 mM L-arginine(HCl) and 25 mM L-lysine(HCl); e) 50 μM DTPA; and f) 0.02% polysorbate 80.

In one aspect, the disclosure provides an anti-RSV antibody formulation comprising: a) about 50 mg/mL to about 250 mg/mL of an anti-RSV antibody, or antigen-binding fragment thereof; b) about 5 mM to about 20 mM histidine; c) about 4% to about 8% (w/v) sucrose; d) an excipient selected from the group consisting of about 25 mM to about 75 mM of L-arginine or a pharmaceutically acceptable salt of L-arginine, about 25 mM to about 75 mM of L-lysine or a pharmaceutically acceptable salt of L-lysine, a mixture of about 25 mM to about 75 mM L-arginine or a pharmaceutically acceptable salt of L-arginine and about 25 mM to about 75 mM L-glutamate or a pharmaceutically acceptable salt of L-glutamate, and a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) about 25 mM to about 75 mM of L-arginine or a pharmaceutically acceptable salt of L-arginine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 20 mM histidine; c) 4% to 8% (w/v) sucrose; d) 25 mM to 75 mM of L-arginine or a pharmaceutically acceptable salt of L-arginine; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) about 25 mM to about 75 mM of L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 20 mM histidine; c) 4% to 8% (w/v) sucrose; d) 25 mM to 75 mM of L-lysine or a pharmaceutically acceptable salt of L-lysine; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) a mixture of about 25 mM to about 75 mM L-arginine or a pharmaceutically acceptable salt of L-arginine and about 25 mM to about 75 mM L-glutamate or a pharmaceutically acceptable salt of L-glutamate; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof; b) 5 mM to 20 mM histidine; c) 4% to about 8% (w/v) sucrose; d) a mixture of 25 mM to 75 mM L-arginine or a pharmaceutically acceptable salt of L-arginine and 25 mM to 75 mM L-glutamate or a pharmaceutically acceptable salt of L-glutamate; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 20 mM histidine; c) 4% to 8% (w/v) sucrose; d) a mixture of 25 mM to 75 mM of L-arginine or a pharmaceutically acceptable salt of L-arginine and 25 mM to 75 mM L-lysine or a pharmaceutically acceptable salt of L-lysine; and e) 0.01% to 0.10% (w/v) polysorbate 80.

In certain embodiments, the formulation has a pH between 5.5 and 6.5.

In certain embodiments, the formulation has a pH of 6.0.

Also provided herein are methods of preventing and/or treating RSV-related diseases in a human patient in need thereof comprising: administering an effective amount of the anti-RSV antibody formulations of the invention to the patient.

Also provided herein are methods of treating or preventing RSV infection in a human patient in need thereof comprising: administering an effective amount of the anti-RSV antibody formulations of the invention to the patient.

Also provided herein are methods of treating or preventing RSV infection in a human patient in need thereof comprising: administering an effective amount of the anti-RSV antibody formulations of the invention to the patient wherein the effective amount comprises a fixed dose of an anti-RSV antibody or antigen-binding fragment thereof, wherein the fixed dose ranges from about 10 to about 100 mg.

Also provided herein are methods of treating or preventing RSV infection in a human patient in need thereof comprising: administering an effective amount of the anti-RSV antibody formulations of the invention to the patient wherein the anti-RSV antibody formulations are administered by subcutaneous administration; or wherein the anti-RSV antibody formulations are administered by intravenous administration; or wherein the anti-RSV antibody formulations are administered by intramuscular administration.

Also provided herein is the use of the anti-RSV antibody formulations of the invention for the treatment or prevention of RSV infection in a human patient.

### BRIEF DESCRIPTION OF THE DRAWINGS

FIGS. 1A-1C show results of UPSEC % HMW, % mAb (monomer) and % LMW vs. Time Data of an anti-RSV antibody formulations at 5° C. storage conditions.

FIGS. 2A-2C show results of UPSEC % HMW, % mAb (monomer) and % LMW vs. Time Data of an anti-RSV antibody formulations at 25° C. storage conditions.

FIGS. 3A-3C show results of UPSEC % HMW, % mAb (monomer) and % LMW vs. Time Data of an anti-RSV antibody formulations at 40° C. storage conditions.

FIGS. 4A-4B show results of HP-IEX % Total Acidic Peaks and % Main Peak vs. Time of an anti-RSV antibody formulations at 5° C. storage conditions.

FIGS. 5A-5B show results of HP-IEX % Total Acidic Peaks and % Main Peak vs. Time of an anti-RSV antibody formulations at 25° C. storage conditions.

FIGS. 6A-6B show results of HP-IEX % Total Acidic Peaks and % Main Peak vs. Time of an anti-RSV antibody formulations at 40° C. storage conditions.

FIG. 7 shows results of excipient reducing viscosity of 100 mg/mL anti-RSV antibody (all experiments were repeated n=5 to derive a standard deviation).

FIGS. 8A-8C show results of UPSEC % HMW, % mAb (monomer) and % LMW vs. Time Data of an anti-RSV antibody formulations at 5° C. storage conditions.

FIGS. 9A-9C show results of UPSEC % HMW, % mAb (monomer) and % LMW vs. Time Data of an anti-RSV antibody formulations at 25° C. storage conditions.

FIGS. 10A-10C show results of UPSEC % HMW, % mAb (monomer) and % LMW vs. Time Data of an anti-RSV antibody formulations at 40° C. storage conditions.

FIGS. 11A-11B show results of HP-IEX % Total Acidic Peaks and % Main Peak vs. Time of an anti-RSV antibody formulations at 5° C. storage conditions.

FIGS. 12A-12B show results of HP-IEX % Total Acidic Peaks and % Main Peak vs. Time of an anti-RSV antibody formulations at 25° C. storage conditions.

FIGS. 13A-13B show results of HP-IEX % Total Acidic Peaks and % Main Peak vs. Time of an anti-RSV antibody formulations at 40° C. storage conditions.

FIG. 14 shows results of the percentage degradation of PS80 at 40° C.

FIG. 15 shows a bar graph comparing viscosity of formulations using excipients of 80 mM histidine, 70 mM lysine or 70 mM arginine.

### DETAILED DESCRIPTION OF THE INVENTION

The invention provides stable formulations comprising an anti-RSV antibody, or antigen-binding fragment thereof that binds to RSV. The invention provides stable formulations comprising an anti-RSV antibody, or antigen-binding fragment thereof that binds to human RSV F-protein, which are useful for methods of treating or preventing human RSV infection.

Antibodies useful in the formulations disclosed herein are described in U.S. Pat. No. 9,963,500. An exemplary antibody, useful in the formulations disclosed herein, is described in U.S. Pat. No. 9,963,500. This exemplary antibody comprises complementarity-determining regions (CDRs) having the amino acid sequences: SEQ ID NO: 1 (heavy chain CDR 1), SEQ ID NO: 2 (heavy chain CDR 2), SEQ ID NO: 3 (heavy chain CDR 3), SEQ ID NO: 4 (light chain CDR 1), SEQ ID NO: 5 (light chain CDR 2), and SEQ ID NO: 6 (light chain CDR 3). Further, this exemplary antibody comprises heavy and light chain variable regions having the amino acid sequences of SEQ ID NO: 7 and SEQ ID NO:8. Further, this exemplary antibody comprises a heavy and light chain immunoglobulin consisting of the amino acid sequences of SEQ ID NO: 9 and SEQ ID NO: 10.

### I. Definitions and Abbreviations

Definitions are utilized throughout the specification. Abbreviations are defined throughout the specification and claims.

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used throughout the specification and in the appended claims, the singular forms “a,” “an,” and “the” include the plural reference unless the context clearly dictates otherwise.

Reference to “or” indicates either or both possibilities unless the context clearly dictates one of the indicated possibilities. In some cases, “and/or” was employed to highlight either or both possibilities.

When a range of values is recited, such as “an amount between 50 mg and 100 mg” the range is intended to be inclusive of the recited values.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell.

“RSV-related disease” means any disease caused, directly or indirectly, by an infection with Respiratory Syncytial Virus (RSV) as well as diseases or conditions which predispose a patient to infection by RSV. Examples of diseases falling into the former category include pneumonia and bronchiolitis. Diseases and conditions in the latter category (i.e., those which place the patient at risk of severe RSV infection) include cystic fibrosis, congenital heart disease, cancer, age related immunosuppression, transplant recipients and, generally, any condition that causes a state of immunosuppression or decreased function of the immune system such as post-operative organ transplantation regimens or premature birth.

“Treat” or “treating” means to administer a therapeutic agent, such as a formulation containing any of the antibodies or antigen-binding fragments of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. Treatment with anti-RSV antibodies could also be combined with other interventions (antibodies, nucleic acids, vaccines and small molecule compounds) to treat other respiratory pathogens.

“Prevent” or “preventing” means to administer a prophylactic agent, such as a formulation containing any of the antibodies or antigen-binding fragments of the present invention, internally or externally to a subject or patient at risk of becoming infected by RSV, for which the agent has prophylactic activity. Preventing includes reducing the likelihood or severity of a subsequent RSV infection, ameliorating symptoms associated with lower respiratory tract infection (LRI) upon RSV infection, and inducing immunity to protect against RSV infection. Typically, the agent is administered in an amount effective to neutralize RSV in the lungs and/or the nose in order block infection. The amount of a prophylactic agent that is effective to ameliorate any particular disease symptom may vary according to factors such as the age, and weight of the patient, and the ability of the agent to elicit a desired response in the subject. Whether a disease symptom has been ameliorated can be assessed by any clinical measurement typically used by physicians or other skilled healthcare providers to assess the severity or progression status of that symptom or in certain instances will ameliorate the need for hospitalization.

The term “patient” (alternatively referred to as “subject” or “individual” herein) refers to a mammal (e.g., rat, mouse, dog, cat, rabbit) capable of being treated with the formulations of the invention, most preferably a human. In some embodiments, the patient is an adult patient. In other embodiments, the patient is a pediatric patient. Those “in need of treatment” include those patients that may benefit from treatment with the formulations of the invention.

The term “pharmaceutically effective amount” or “effective amount” means an amount whereby a sufficient therapeutic formulation is introduced to a patient to treat a disease or condition. One skilled in the art recognizes that this level may vary according the patient's characteristics such as age, weight, etc.

As used herein, the phrase “fixed dose” refers to an amount (e.g. in milligrams) of active ingredient that is administered to a patient.

The term “about”, when modifying the quantity (e.g., mM, or M) of a substance or formulation, the percentage (v/v or w/v) of a formulation component, the pH of a solution/formulation, or the value of a parameter characterizing a step in a method, or the like, refers to variation in the numerical quantity of plus or minus 5%. Such variation in the numerical quantity can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like.

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or formulation. As a non-limiting example, a binding compound that consists essentially of a recited amino acid sequence may also include one or more amino acids, including substitutions of one or more amino acid residues, that do not materially affect the properties of the binding compound.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication. The phrase “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout.

The phrase “Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, in one embodiment, a stable formulation is a formulation with no significant changes observed at a refrigerated temperature (2-8° C.) for at least 12 months. In another embodiment, a stable formulation is a formulation with no significant changes observed at a refrigerated temperature (2-8° C.) for at least 18 months. In another embodiment, stable formulation is a formulation with no significant changes observed at room temperature (23-27° C.) for at least 3 months. In another embodiment, stable formulation is a formulation with no significant changes observed at room temperature (23-27° C.) for at least 6 months. In another embodiment, stable formulation is a formulation with no significant changes observed at room temperature (23-27° C.) for at least 12 months. In another embodiment, stable formulation is a formulation with no significant changes observed at room temperature (23-27° C.) for at least 18 months. The criteria for stability for an antibody formulation are as follows. Typically, no more than 10%, preferably 5%, of antibody monomer is degraded as measured by SEC-HPLC. Typically, the formulation is colorless, or clear to slightly opalescent by visual analysis. Typically, the concentration, pH and osmolality of the formulation have no more than +/−10% change. Potency is typically within 60-140%, preferably 80-120%, of the control or reference. Typically, no more than 10%, preferably 5%, of clipping of the antibody is observed, i.e., % low molecular weight species as determined, for example, by HP-SEC. Typically, no more than 10%, preferably no more than 5%, of aggregation of the antibody is observed, i.e. % high molecular weight species as determined, for example, by HP-SEC.

The phrase “anti-RSV antibody” refers to a monoclonal antibody directed against the F protein or the G protein of RSV. Anti-RSV antibodies are disclosed and described in U.S. Pat. No. 9,963,500. A particular anti-RSV antibody is disclosed and described in U.S. Pat. No. 9,963,500 and comprises complementarity-determining regions (CDRs) having the amino acid sequences: SEQ ID NO: 1 (heavy chain CDR 1), SEQ ID NO: 2 (heavy chain CDR 2), SEQ ID NO: 3 (heavy chain CDR 3), SEQ ID NO: 4 (light chain CDR 1), SEQ ID NO: 5 (light chain CDR 2), and SEQ ID NO: 6 (light chain CDR 3). Further, an anti-RSV antibody is disclosed and described in U.S. Pat. No. 9,963,500 and comprises heavy and light chain variable regions having the amino acid sequences of SEQ ID NO: 7 and SEQ ID NO: 8, respectively. Further, a particular anti-RSV antibody is disclosed and described in U.S. Pat. No. 9,963,500 and comprises a heavy and light chain immunoglobulin consisting of the amino acid sequences of SEQ ID NO: 9 and SEQ ID NO: 10, respectively.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC) and dynamic light scattering. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen-binding assay. Formulations of the invention include antibodies and fragments thereof that are biologically active when reconstituted or in liquid form.

### II. Formulations of the Invention

The formulations of the invention minimize the formation of antibody aggregates, increase stability and reduce viscosity.

The invention includes various formulations of an anti-RSV antibody, or antigen-binding fragment thereof, as described in more detail below. For example, the invention includes formulations comprising (i) an anti-RSV antibody or antigen-binding fragment thereof, (ii) a buffer (e.g., histidine), (iii) a stabilizer (e.g., a non-reducing sugar such as sucrose); (iv) a non-ionic surfactant (e.g., polysorbate 80); and (v) an antioxidant (e.g., methionine). In further embodiments, the formulations of the invention comprise a viscosity-reducer (e.g. arginine, or a pharmaceutically acceptable salt thereof; lysine, or a pharmaceutically acceptable salt thereof, a mixture of arginine and lysine or pharmaceutically acceptable salts thereof, a mixture of arginine and glutamate or pharmaceutically acceptable salts thereof, and/or histidine or pharmaceutically acceptable salts thereof) and/or a metal chelator (e.g. DTPA).

### III. Anti-RSV Antibodies and Antigen-Binding Fragments Thereof

The invention provides stable biological formulations comprising an anti-RSV antibody which comprises complementarity-determining regions (CDRs) having the amino acid sequences: SEQ ID NO: 1 (heavy chain CDR 1), SEQ ID NO: 2 (heavy chain CDR 2), SEQ ID NO: 3 (heavy chain CDR 3), SEQ ID NO: 4 (light chain CDR 1), SEQ ID NO: 5 (light chain CDR 2), and SEQ ID NO: 6 (light chain CDR 3). The invention also provides stable biological formulations comprising an anti-RSV antibody which comprises heavy and light chain variable regions having the amino acid sequences of SEQ ID NO: 7 and SEQ ID NO: 8, respectively and/or comprises a heavy and light chain immunoglobulin consisting of the amino acid sequences of SEQ ID NO: 9 and SEQ ID NO: 10 respectively. The methods of making the anti-RSV antibody are disclosed and described in U.S. Pat. No. 9,963,500 and are hereby incorporated by reference in its entirety. SEQ ID NOs: 1-10 are set forth in Table A below:

TABLE A
Sequence Information

| SEQ | |
|---|---|
| ID | |
| NO: | SEQUENCE |
| 1 | DSAMS |
| 2 | FIKSKTYGGTKEYAASVKG |
| 3 | GAPYGGNSDYYYGLDV |
| 4 | RTSQDVRGALA |
| 5 | DASSLET |
| 6 | QQFLDFPFT |
| 7 | EVQLVESGGGLVRPGRSLRLSCTVSGFSFDDSAMSWVRQAPG |
| | KGLEWISFIKSKTYGGTKEYAASVKGRFTISRDDSKNIAYLQ |
| | MNSLKTEDTAVYYCTRGAPYGGNSDYYYGLDVWGQGTTVT |
| 8 | MTQSPSSLSASVGDRVTITCRTSQDVRGALAWYQQKPGKAPK |
| | LLIFDASSLETGVPSRFSGSGSGTVFTLTISSLQPEDFAAYY |
| | CQQFLDFPFTFGQGTRLEIKRT |
| 9 | EVQLVESGGGLVRPGRSLRLSCTVSGFSFDDSAMSWVRQAPG |
| | KGLEWISFIKSKTYGGTKEYAASVKGRFTISRDDSKNIAYLQ |
| | MNSLKTEDTAVYYCTRGAPYGGNSDYYYGLDVWGQGTTVTVS |
| | SASTKGPSVFPLAPSSKSISGGTAALGCLVKDYFPEPVTVSW |
| | NSGALTSGVHTFPAVLQSSGLYSLSSVVTVPSSSLGTQTYIC |
| | NVNHKPSNTKVDKKVEPKSCDKTHTCPPCPAPELLGGPSVFL |
| | FPPKPKDTLYITREPEVTCVVVDVSHEDPEVKFNWYVDGVEV |
| | HNAKTKPREEQYNSTYRVVSVLTVLHQDWLNGKEYKCKVSNK |
| | ALPAPIEKTISKAKGQPREPQVYTLPPSRDELTKNQVSLTCL |
| | VKGFYPSDIAVEWESNGQPENNYKTTPPVLDSDGSFFLYSKL |
| | TVDKSRWQQGNVFSCSVMHEALHNHYTQKSL SLSPGK |
| 10 | DIQMTQSPSSLSASVGDRVTITCRTSQDVRGALAWYQQKPGK |
| | APKLLIFDASSLETGVPSRFSGSGSGTVFTLTISSLQPEDFA |
| | AYYCQQFLDFPFTFGQGTRLEIKRTVAAPSVFIFPPSDEQLK |
| | SGTASVVCLLNNFYPREAKVQWKVDNALQSGNSQESVTEQDS |
| | KDSTYSLSSTLTLSKADYEKHKVYACEVTHQGLSSPVTKSFN |
| | RGEC |

In some embodiments of the formulations described herein, the API (“active pharmaceutical ingredient”, i.e. the anti-RSV antibody or antigen-binding fragment thereof) is present in a concentration of about 50 mg/mL to about 250 mg/mL. In some embodiments of the formulations, the API (i.e. the anti-RSV antibody or antigen-binding fragment thereof) is present in a concentration of about 75 mg/mL to about 200 mg/mL. In some embodiments, the API is present in a concentration of about 90 mg/mL to about 110 mg/mL. In another embodiment, the API is present in a concentration of about 100 mg/mL. In another embodiment, the API is present in a concentration of 90 mg/mL to 110 mg/mL. In another embodiment, the API is present in a concentration of 100 mg/mL. In another embodiment, the API is present in a concentration of 75 mg/mL. In another embodiment, the API is present in a concentration of 50 mg/mL.

In some embodiments, the API is present in a concentration of about 125 mg/mL to about 175 mg/mL. In some embodiments, the API is present in a concentration of about 140 mg/mL to about 160 mg/mL. In another embodiment, the API is present in a concentration of about 150 mg/mL. In some embodiments, the API is present in a concentration of 125 mg/mL to 175 mg/mL. In some embodiments, the API is present in a concentration of 140 mg/mL to 160 mg/mL. In another embodiment, the API is present in a concentration of 150 mg/mL.

In some embodiments, the API is present in a concentration of about 150 mg/mL to about 200 mg/mL. In some embodiments, the API is present in a concentration of about 165 mg/mL to about 185 mg/mL. In another embodiment, the API is present in a concentration of about 175 mg/mL. In some embodiments, the API is present in a concentration of 150 mg/mL to 200 mg/mL. In some embodiments, the API is present in a concentration of 165 mg/mL to 185 mg/mL. In another embodiment, the API is present in a concentration of 175 mg/mL.

In some embodiments, the API is present in a concentration of about 175 mg/mL to about 225 mg/mL. In some embodiments, the API is present in a concentration of about 190 mg/mL to about 210 mg/mL. In another embodiment, the API is present in a concentration of about 200 mg/mL. In some embodiments, the API is present in a concentration of 175 mg/mL to 225 mg/mL. In some embodiments, the API is present in a concentration of 190 mg/mL to 210 mg/mL. In another embodiment, the API is present in a concentration of 200 mg/mL.

In some embodiments, the API is present in a concentration of about 200 mg/mL to about 250 mg/mL. In some embodiments, the API is present in a concentration of about 215 mg/mL to about 235 mg/mL. In another embodiment, the API is present in a concentration of about 225 mg/mL. In some embodiments, the API is present in a concentration of 200 mg/mL to 250 mg/mL. In some embodiments, the API is present in a concentration of 215 mg/mL to 235 mg/mL. In another embodiment, the API is present in a concentration of 225 mg/mL.

In some embodiments of the formulations disclosed herein, the API (i.e. the anti-RSV antibody or antigen-binding fragment thereof) is present in a fixed dose (e.g. an amount in milligrams). In some embodiments of the formulations disclosed herein, the API is present in a fixed dose of about 10 mg to about 150 mg. In some embodiments, the API is present in a fixed dose of 10 mg to 150 mg. In some embodiments, the API is present in a fixed dose of about 25 mg to about 125 mg. In some embodiments, the API is present in a fixed dose of 25 mg to 125 mg. In some embodiments, the API is present in a fixed dose of about 50 mg to about 100 mg. In some embodiments, the API is present in a fixed dose of 50 mg to 100 mg.

### IV. Formulation Excipients

The formulations of the invention comprise at least one excipient that stabilizes the formulation.

In some embodiments of the formulations of the invention, the stabilizer is a non-reducing sugar. In embodiments of the invention, the non-reducing sugar is sucrose. In further embodiments, the non-reducing sugar is glucose. In additional embodiments, the non-reducing sugar is trehalose. In still further embodiments, the non-reducing sugar is lactose. In other embodiments, the non-reducing sugar is raffinose.

In some embodiments, the anti-RSV antibody formulations of the invention comprise a stabilizer selected from the group consisting of: about 4% to about 8% weight/volume (w/v) sucrose, glucose, trehalose, lactose or raffinose.

In some embodiments, the stabilizer is about 4% to about 8% w/v sucrose. In some embodiments, the stabilizer is 4% to 8% w/v sucrose.

In some embodiments, the stabilizer is about 6% to about 8% w/v sucrose.

In some embodiments, the stabilizer is 4% to 8% w/v sucrose.

In some embodiments, the stabilizer is about 4% to about 8% w/v trehalose. In some embodiments, the stabilizer is 4% to 8% w/v trehalose.

In some embodiments, the stabilizer is about 6% to about 8% w/v trehalose. In some embodiments, the stabilizer is 6% to 8% w/v trehalose.

In some embodiments, the stabilizer is about 6.5% to about 7.5% w/v sucrose. In some embodiments, the stabilizer is 6.5% to 7.5% w/v sucrose.

In some embodiments, the stabilizer is 6% to 8% sucrose.

In some embodiments, the stabilizer is 6.5% to 7.5% w/v sucrose.

In some embodiments, the stabilizer is 7% sucrose.

The formulations of the invention comprise arginine, e.g., L-arginine, or a pharmaceutically acceptable salt thereof, e.g., HCl; lysine, e.g., L-lysine, or a pharmaceutically acceptable salt thereof, e.g., HCl; or a combination of arginine (L-arginine), or a pharmaceutically acceptable salt (HCl) thereof and lysine (L-lysine), or a pharmaceutically acceptable salt (HCl) thereof, or a combination of arginine (L-arginine), or a pharmaceutically acceptable salt (HCl) thereof and glutamate (L-glutamate), or a pharmaceutically acceptable salt thereof, all of which may provide additional stability to the formulation, as well as control viscosity, which allows formulation at high API concentration.

In addition to an anti-RSV antibody or antigen-binding fragment thereof, and a stabilizer in the amounts/concentrations specified above, the formulations of the invention also comprise a buffer. In some embodiments, the buffer is present in an amount of about 5 mM to about 20 mM, which provides for a pH in the range of about 5 to about 7. In some embodiments, the buffer is present in an amount of 5 mM to 20 mM, which provides for a pH in the range of 5 to 7.

In some embodiments of the invention, the buffer provides the formulation a pH in the range from about 5.5 to about 6.5. In some embodiments of the invention, the buffer provides the formulation a pH in the range from 5.5 to 6.5. In some embodiments, the buffer has a pH in a range of about 6.0. In still further embodiments, the pH is 6.0.

In particular embodiments, the buffer has a pH of about 5.7, about 5.8, about 5.9, about 6.0, about 6.1, about 6.2, or about 6.3. In particular embodiments, the buffer has a pH of 5.7, 5.8, 5.9, 6.0, 6.1, 6.2, or 6.3. Examples of buffers that will control the pH in this range include succinate (sodium or potassium), histidine, sodium acetate, phosphate (sodium or potassium), Tris (tris (hydroxymethyl) aminomethane), diethanolamine, citrate (sodium) and other organic acid buffers.

In some embodiments of the invention, the buffer is histidine or acetate at a pH of about 5.5 to about 6.5. In some embodiments of the invention, the buffer is histidine or acetate at a pH of 5.5 to 6.5. In some embodiments, the buffer is an L-histidine buffer. In embodiments where the formulation is lyophilized, it is preferred that the buffer is not acetate because acetate buffer systems are not compatible with the lyophilization process.

When a range of pH values is recited, such as “a pH between 5.5 and 6.5,” the range is intended to be inclusive of the recited values. Unless otherwise indicated, for lyophilized formula the pH refers to the pH after reconstitution of the lyophilized formulations of the invention. The pH is typically measured at 25° C. using standard glass bulb pH meter. As used herein, a solution comprising “histidine buffer at pH X” refers to a solution at pH X and comprising the histidine buffer, i.e. the pH is intended to refer to the pH of the solution.

In addition to an anti-RSV antibody or antigen-binding fragment thereof, a stabilizer, and a buffer in the amounts/concentrations specified above, the formulations of the invention also comprise an anti-oxidant. In embodiments of the invention, the anti-oxidant is methionine. In embodiments of the invention, the anti-oxidant is L-methionine, or a pharmaceutically acceptable salt thereof. In further embodiments, the methionine is L-methionine. In other embodiments, the anti-oxidant is L-methionine HCl. In other embodiments, the anti-oxidant is histidine.

In some embodiments, the anti-oxidant (e.g. L-methionine) is present in the formulations of the invention in an amount of about 1 mM to about 20 mM. In some embodiments, the anti-oxidant is present in an amount of about 5 mM to about 20 mM, about 5 mM to about 15 mM, about 5 mM to about 10 mM. In some embodiments, the anti-oxidant is present in an amount of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM. In some embodiments, the anti-oxidant is present in an amount of 5 mM to 20 mM, 5 mM to 15 mM, 5 mM to 10 mM. In some embodiments, the anti-oxidant is present in an amount of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM or 20 mM.

In embodiments wherein the anti-oxidant is histidine, the histidine can be present in amounts up to 100 mM. In such embodiments, histidine can serve as a buffer, an anti-oxidant, and/or to reduce viscosity in the formulations described herein. In some embodiments, histidine may be present in a concentration of about 10-20 mM, about 20-30 mM, about 30-40 mM, about 40-50 mM, about 50-60 mM, about 60-70 mM, about 70-80 mM, about 80-90 mM, or about 90-100 mM. In some embodiments, histidine may be present in a concentration of 10-20 mM, 20-30 mM, 30-40 mM, 40-50 mM, 50-60 mM, 60-70 mM, 70-80 mM, 80-90 mM, or 90-100 mM. In some embodiments, histidine may be present in a concentration of about 10 mM, about 20 mM, about 30 mM, about 40 mM, about 50 mM, about 60 mM, about 70 mM, about 80 mM, about 90 mM, or about 100 mM. In some embodiments, histidine may be present in a concentration of 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In addition to an anti-RSV antibody or antigen-binding fragment thereof, a stabilizer, a buffer, and an anti-oxidant in the amounts/concentrations specified above, the formulations of the invention also comprise a surfactant. Surfactants are typically added to formulations to provide stability, reduce and/or prevent aggregation or to prevent and/or inhibit protein damage during processing conditions such as purification, filtration, freeze-drying, transportation, storage, and delivery. In some embodiments of the invention, a surfactant is useful for providing additional stability to the active ingredient(s), i.e. the anti-RSV antibody or antigen-binding fragment thereof.

Surfactants that may be useful in the formulations of the invention include, but are not limited to: nonionic surfactants such as polyoxyethylene sorbitan fatty acid esters (Polysorbates, sold under the trade name TWEEN (Uniquema Americas LLC, Wilmington, DE)) including Polysorbate-20 (polyoxyethylene sorbitan monolaurate), Polysorbate-40 (polyoxyethylene sorbitan monopalmitate), Polysorbate-60 (polyoxyethylene sorbitan monostearate), and Polysorbate-80 (polyoxyethylene sorbitan monooleate); polyoxyethylene alkyl ethers such as BRIJ 58 (Uniquema Americas LLC, Wilmington, DE) and BRIJ 35; poloxamers (e.g., poloxamer 188); TRITON X-100 (Union Carbide Corp., Houston, TX) and TRITON X-114; NP40; Span 20, Span 40, Span 60, Span 65, Span 80 and Span 85; copolymers of ethylene and propylene glycol (e.g., the PLURONIC series of nonionic surfactants such as PLURONIC F68, PLURONIC 10R5, PLURONIC F108, PLURONIC F127, PLURONIC F38, PLURONIC L44, PLURONIC L62 (BASF Corp., Ludwigshafen, Germany); and sodium dodecyl sulfate (SDS).

The amount of surfactant to be included in the formulations of the invention is an amount sufficient to perform the desired function, i.e. a minimal amount necessary to stabilize the active pharmaceutical ingredient (i.e. the anti-RSV antibody or antigen-binding fragment thereof) in the formulation. Typically, the surfactant is present in a concentration of from about 0.008% to about 0.1% w/v. In some embodiments of this aspect of the invention, the surfactant is present in the formulation in an amount from about 0.01% to about 0.04% w/v; from about 0.01% to about 0.03% w/v, from about 0.01% to about 0.02% w/v, from about 0.015% to about 0.04% w/v; from about 0.015% to about 0.03% w/v, from about 0.015% to about 0.02% w/v, from about 0.02% to about 0.04% w/v, from about 0.02% to about 0.035% w/v, or from about 0.02% to about 0.03% w/v. In specific embodiments, the surfactant is present in an amount of about 0.02% w/v. In alternative embodiments, the surfactant is present in an amount of about 0.01%, about 0.015%, about 0.025%, about 0.03%, about 0.035%, or about 0.04% w/v.

In exemplary embodiments of the invention, the surfactant is a nonionic surfactant selected from the group consisting of. Polysorbate 20, Polysorbate 80 and F127. In preferred embodiments, the surfactant is Polysorbate 80.

In specific embodiments, the anti-RSV antibody formulations comprise about 0.01% to about 0.04% w/v PS80. In further embodiments, the anti-RSV antibody formulations comprise PS80 in an amount of about 0.008%, about 0.01%, about 0.015%, about 0.02%, about 0.025%, about 0.03%, about 0.035%, about 0.04% or about 0.045% w/v. In particular embodiments, the anti-RSV antibody formulations comprise about 0.02% w/v PS80. In some embodiments, the anti-RSV antibody formulations comprise 0.01% to 0.04% w/v PS80. In some embodiments, the anti-RSV antibody formulations comprise PS80 in an amount of 0.008%, 0.01%, 0.015%, 0.02%, 0.025%, 0.03%, 0.035%, 0.04% or 0.045% w/v. In particular embodiments, the anti-RSV antibody formulations comprise 0.02% w/v PS80.

In specific embodiments, the anti-RSV antibody formulations of the invention comprise a chelator selected from DTPA and EDTA. In some embodiments, the chelator is DTPA. In further embodiments, the amount of DTPA is about 10 μM to about 90 μM, about 25 μM to about 75 μM, or about 50 μM. In some embodiments, the chelator is DTPA. In some embodiments, the amount of DTPA is 10 μM to 90 μM, 25 μM to 75 μM, or 50 μM.

In addition to an anti-RSV antibody or antigen-binding fragment thereof, embodiments of the formulation can contain a stabilizer, a buffer, an anti-oxidant, a surfactant, and a chelator, in the amounts/concentrations specified above.

The disclosure also provides an anti-RSV antibody formulation as described herein, wherein the formulation is contained in a glass vial or injection device (e.g. a syringe).

In further embodiments, the disclosure provides anti-RSV antibody formulations as described herein, wherein after storage of the formulation at 2-8° C. for 12 months, the percent of heavy chain and light chain measured by reducing CE-SDS is >96%.

In further embodiments, the disclosure provides anti-RSV antibody formulations as described herein, wherein after storage of the formulation at 2-8° C. for 12 months the percent of intact IgG in the formulation measured by non-reducing CE-SDS is >97%.

In further embodiments, the disclosure provides anti-RSV antibody formulations as described herein, wherein after storage of the formulation at 2-8° C. for 12 months, the percent of monomer as measured by HP-SEC is >98.5%.

In additional embodiments, the disclosure provides anti-RSV antibody formulations as described herein, wherein after storage of the formulation at 2-8° C. for 12 months, the percent of high molecular weight species as measured by HP-SEC is <1.5%.

In further embodiments, the disclosure provides anti-RSV antibody formulations as described herein, wherein after storage of the formulation at 25° C. for 12 months, the percent of monomer as measured by HP-SEC is >98.0%.

In additional embodiments, the disclosure provides anti-RSV antibody formulations as described herein, wherein after storage of the formulation at 25° C. for 6 months, the percent of high molecular weight species as measured by HP-SEC is <2%.

In further embodiments, the disclosure provides anti-RSV antibody formulations as described herein, wherein after storage of the formulation at 40° C. for 3 months, the percent of monomer as measured by HP-SEC is >94.0%, >94.5% or >95.0%.

In additional embodiments, the disclosure provides anti-RSV antibody formulations as described herein, wherein after storage of the formulation at 40° C. for 3 months, the percent of high molecular weight species as measured by HP-SEC is <5.5%, <5.0%, or <4.4%.

### V. Specific Aspects and Embodiments of the Invention

In one aspect, the present disclosure provides an anti-RSV antibody formulation, comprising: a) about 50 mg/mL to about 250 mg/mL of an anti-RSV antibody, or antigen-binding fragment thereof; b) about 5 mM to about 20 mM buffer; c) a stabilizer selected from the group consisting of: (i) about 6% to about 8% weight/volume (w/v) non-reducing sugar; (ii) about 25 mM to about 75 mM of L-arginine, or a pharmaceutically acceptable salt of L-arginine; about 25 mM to about 75 mM L-proline, or a pharmaceutically acceptable salt of L-proline; about 25 mM to about 75 mM L-glutamate, or a pharmaceutically acceptable salt of L-glutamate; about 25 mM to about 75 mM L-lysine, or a pharmaceutically acceptable salt of L-lysine, or a mixture of such amino acids, and (iii) about 25 μM to 75 μM of a chelator; d) about 0.01% to about 0.10% of a non-ionic surfactant; and optionally e) about 1 mM to about 20 mM of an anti-oxidant.

In another aspect, the disclosure provides an anti-RSV antibody formulation, comprising: a) about 50 mg/mL to about 150 mg/mL of an anti-RSV antibody, or antigen-binding fragment thereof, b) about 5 mM to about 20 mM histidine; c) about 6% to about 8% weight/volume (w/v) sucrose; d) an excipient selected from about 25 mM to about 75 mM of L-arginine or a pharmaceutically acceptable salt of L-arginine, about 25 mM to about 75 mM L-lysine or a pharmaceutically acceptable salt of L-lysine, about 25 mM to about 75 mM of a mixture of L-arginine(HCl) and L-glutamate, and about 25 mM to about 75 mM of a mixture of L-arginine(HCl) and L-lysine(HCl); e) about 25 to 75 μM of DTPA; f) about 0.01% to about 0.10% polysorbate 80; and optionally g) about 1 mM to about 20 mM L-methionine.

In another aspect, the disclosure provides an anti-RSV antibody formulation, comprising: a) 100 mg/mL of an anti-RSV antibody, or antigen-binding fragment thereof, b) 10 mM histidine; c) 7% weight/volume (w/v) sucrose; d) an excipient selected from 50 mM L-arginine(HCl), 50 mM L-lysine(HCl), a mixture of 25 mM L-arginine and 25 mM L-glutamate, and a mixture of 25 mM L-arginine(HCl) and 25 mM L-lysine(HCl); e) 50 μM DTPA; f) 0.02% polysorbate 80; and optionally g) 1 mM to 20 mM L-methionine.

In another aspect, the disclosure provides an anti-RSV antibody formulation, comprising: a) 100 mg/mL of an anti-RSV antibody, or antigen-binding fragment thereof, b) 10 mM histidine; c) 7% weight/volume (w/v) sucrose; d) a mixture of 25 mM L-arginine(HCl) and 25 mM L-lysine(HCl); e) 50 μM DTPA; and f) 0.02% polysorbate 80.

In another aspect, the concentration of the anti-RSV antibody, or antigen-binding fragment thereof, is about 50 mg/mL to about 150 mg/mL. In another aspect, the concentration of the anti-RSV antibody, or antigen-binding fragment thereof, is about 75 mg/mL to about 125 mg/mL. In another aspect, the concentration of the anti-RSV antibody, or antigen-binding fragment thereof, is 50 mg/mL to 150 mg/mL. In another aspect, the concentration of the anti-RSV antibody, or antigen-binding fragment thereof, is 75 mg/mL to 125 mg/mL. In another aspect, the concentration of the anti-RSV antibody, or antigen-binding fragment thereof, is 100 mg/mL. In another aspect, the concentration of the anti-RSV antibody, or antigen-binding fragment thereof, is 75 mg/mL. In another aspect, the concentration of the anti-RSV antibody, or antigen-binding fragment thereof, is 50 mg/mL.

In another aspect, the formulation has a pH between about 5.5 and about 6.5. In another aspect, the formulation has a pH between 5.5 and 6.5. In another aspect, the formulation has a pH of 6.0.

In another aspect, the buffer is histidine. In another aspect, the concentration of histidine is from about 1 mM to about 20 mM. In another aspect, the concentration of histidine is from about 5 mM to about 15 mM. In another aspect, the concentration of histidine is 1 mM to 20 mM. In another aspect, the concentration of histidine is 5 mM to 15 mM. In another aspect, the concentration of histidine is 10 mM.

In another aspect, the instant formulation comprises a stabilizer selected from a non-reducing sugar; L-arginine, or a pharmaceutically acceptable salt of L-arginine; L-proline, or a pharmaceutically acceptable salt of L-proline; L-glutamate, or a pharmaceutically acceptable salt of L-glutamate; L-lysine, or a pharmaceutically acceptable salt of L-lysine; or a mixture of such amino acids. In another aspect, the instant formulation comprises an excipient selected from about 50 mM L-arginine(HCl), about 50 mM L-lysine(HCl), a mixture of about 25 mM L-arginine and about 25 mM L-glutamate, and a mixture of about 25 mM L-arginine(HCl) and about 25 mM L-lysine(HCl).

In another aspect, the instant formulation comprises a chelator which is DTPA at a concentration of about 25 μM to about 75 μM. In another aspect, the instant formulation comprises a chelator which is DTPA at a concentration of about 50 μM. In another aspect, the instant formulation comprises a chelator which is DTPA at a concentration of 50 μM.

In another aspect, the formulation comprises a non-ionic surfactant which is PS80 at a concentration of about 0.01% to about 0.10% w/v. In another aspect, the formulation comprises a non-ionic surfactant which is PS80 at a concentration of about 0.02% w/v. In another aspect, the formulation comprises a non-ionic surfactant which is PS80 at a concentration of 0.02% w/v.

In another aspect, the formulation comprises an antioxidant which is L-methionine at a concentration of about 1 mM to about 20 mM. In another aspect, the formulation comprises an antioxidant which is L-methionine at a concentration of about 10 mM. In another aspect, the formulation comprises an antioxidant which is L-methionine at a concentration of 10 mM.

In yet another aspect, the disclosure provides an anti-RSV antibody formulation comprising: a) about 50 mg/mL to about 250 mg/mL of an anti-RSV antibody, or antigen-binding fragment thereof; b) about 5 mM to about 20 mM histidine; c) about 4% to about 8% (w/v) sucrose; d) an excipient selected from the group consisting of about 25 mM to about 75 mM of L-arginine or a pharmaceutically acceptable salt of L-arginine, about 25 mM to about 75 mM of L-lysine or a pharmaceutically acceptable salt of L-lysine, a mixture of about 25 mM to about 75 mM L-arginine or a pharmaceutically acceptable salt of L-arginine and about 25 mM to about 75 mM L-glutamate or a pharmaceutically acceptable salt of L-glutamate, and a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) about 25 mM to about 75 mM of L-arginine or a pharmaceutically acceptable salt of L-arginine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 20 mM histidine; c) 4% to 8% (w/v) sucrose; d) 25 mM to 75 mM of L-arginine or a pharmaceutically acceptable salt of L-arginine; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) about 25 mM to about 75 mM of L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 20 mM histidine; c) 4% to 8% (w/v) sucrose; d) 25 mM to 75 mM of L-lysine or a pharmaceutically acceptable salt of L-lysine; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) a mixture of about 25 mM to about 75 mM L-arginine or a pharmaceutically acceptable salt of L-arginine and about 25 mM to about 75 mM L-glutamate or a pharmaceutically acceptable salt of L-glutamate; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof; b) 5 mM to 20 mM histidine; c) 4% to about 8% (w/v) sucrose; d) a mixture of 25 mM to 75 mM L-arginine or a pharmaceutically acceptable salt of L-arginine and 25 mM to 75 mM L-glutamate or a pharmaceutically acceptable salt of L-glutamate; and e) 0.01% to 0.10% (w/v) polysorbate 80.

In one embodiment, the anti-RSV antibody formulation comprises: a) about 50 mg/ml to about 250 mg/ml of an anti-RSV antibody, or antigen-binding fragment thereof, b) about 5 mM to about 20 mM histidine; c) about 4% to about 8% (w/v) sucrose; d) a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof; b) 5 mM to 20 mM histidine; c) 4% to 8% (w/v) sucrose; d) a mixture of 25 mM to 75 mM of L-arginine or a pharmaceutically acceptable salt of L-arginine and 25 mM to 75 mM L-lysine or a pharmaceutically acceptable salt of L-lysine; and e) 0.01% to 0.10% (w/v) polysorbate 80.

In still another aspect, the disclosure provides an anti-RSV antibody formulation comprising: a) about 50 mg/mL to about 250 mg/mL of an anti-RSV antibody, or antigen-binding fragment thereof, b) about 5 mM to about 100 mM histidine; c) about 4% to about 8% (w/v) sucrose; and d) about 0.01% to about 0.10% (w/v) polysorbate 80. In one embodiment, the disclosure provides an anti-RSV antibody formulation comprising: a) 50 mg/mL to 250 mg/mL of an anti-RSV antibody, or antigen-binding fragment thereof, b) 5 mM to 100 mM histidine; c) 4% to 8% (w/v) sucrose; and d) 0.01% to 0.10% (w/v) polysorbate 80.

In one aspect, the anti-RSV antibody formulation comprises: a) about 50 mg/ml to about 250 mg/ml of an anti-RSV antibody, or antigen-binding fragment thereof, b) about 5 mM to about 100 mM histidine; c) about 4% to about 8% (w/v) sucrose; d) about 25 mM to about 75 mM of L-arginine or a pharmaceutically acceptable salt of L-arginine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 100 mM histidine; c) 4% to 8% (w/v) sucrose; d) 25 mM to 75 mM of L-arginine or a pharmaceutically acceptable salt of L-arginine; and e) 0.01% to 0.10% (w/v) polysorbate 80.

In one aspect, the anti-RSV antibody formulation comprises: a) about 50 mg/ml to about 250 mg/ml of an anti-RSV antibody, or antigen-binding fragment thereof, b) about 5 mM to about 100 mM histidine; c) about 4% to about 8% (w/v) sucrose; d) about 25 mM to about 75 mM of L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 100 mM histidine; c) 4% to 8% (w/v) sucrose; d) 25 mM to 75 mM of L-lysine or a pharmaceutically acceptable salt of L-lysine; and e) 0.01% to 0.10% (w/v) polysorbate 80.

In one aspect, the anti-RSV antibody formulation comprises: a) about 50 mg/ml to about 250 mg/ml of an anti-RSV antibody, or antigen-binding fragment thereof, b) about 5 mM to about 100 mM histidine; c) about 4% to about 8% (w/v) sucrose; d) a mixture of about 25 mM to about 75 mM L-arginine or a pharmaceutically acceptable salt of L-arginine and about 25 mM to about 75 mM L-glutamate or a pharmaceutically acceptable salt of L-glutamate; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 100 mM histidine; c) 4% to about 8% (w/v) sucrose; d) a mixture of 25 mM to 75 mM L-arginine or a pharmaceutically acceptable salt of L-arginine and 25 mM to 75 mM L-glutamate or a pharmaceutically acceptable salt of L-glutamate; and e) 0.01% to 0.10% (w/v) polysorbate 80.

In one aspect, the anti-RSV antibody formulation comprises: a) about 50 mg/ml to about 250 mg/ml of an anti-RSV antibody, or antigen-binding fragment thereof, b) about 5 mM to about 100 mM histidine; c) about 4% to about 8% (w/v) sucrose; d) a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80. In one embodiment, the anti-RSV antibody formulation comprises: a) 50 mg/ml to 250 mg/ml of an anti-RSV antibody, or antigen-binding fragment thereof, b) 5 mM to 100 mM histidine; c) 4% to 8% (w/v) sucrose; d) a mixture of 25 mM to 75 mM of L-arginine or a pharmaceutically acceptable salt of L-arginine and 25 mM to 75 mM L-lysine or a pharmaceutically acceptable salt of L-lysine; and e) 0.01% to 0.10% (w/v) polysorbate 80.

In certain embodiments of any of the foregoing aspects and embodiments, the histidine concentration is between about 70 mM and about 90 mM. In some embodiments, the histidine concentration is between 70 mM and 90 mM. In some embodiments, the histidine concentration is about 80 mM. In some embodiments, the histidine concentration is 80 mM.

In some embodiments of the above aspects and embodiments, the anti-RSV antibody formulation comprises about 125 mg/mL to about 175 mg/mL of the anti-RSV antibody, or antigen-binding fragment thereof. In one embodiment, the anti-RSV antibody formulation comprises 125 mg/mL to 175 mg/mL of the anti-RSV antibody, or antigen-binding fragment thereof. In one embodiment, the anti-RSV antibody formulation comprises about 150 mg/ml of the anti-RSV antibody, or antigen-binding fragment thereof. In one embodiment, the anti-RSV antibody formulation comprises 150 mg/ml of the anti-RSV antibody, or antigen-binding fragment thereof.

In some embodiments of the above aspects and embodiments, the anti-RSV antibody formulation comprises about 150 mg/mL to about 200 mg/mL of the anti-RSV antibody, or antigen-binding fragment thereof. In some embodiments, the anti-RSV antibody formulation comprises 150 mg/mL to 200 mg/mL of the anti-RSV antibody, or antigen-binding fragment thereof. In some embodiments, the anti-RSV antibody formulation comprises about 175 mg/ml of the anti-RSV antibody, or antigen-binding fragment thereof. In some embodiments, the anti-RSV antibody formulation comprises 175 mg/ml of the anti-RSV antibody, or antigen-binding fragment thereof.

In some embodiments of the above aspects and embodiments, the anti-RSV antibody formulation comprises about 175 mg/mL to about 225 mg/mL of the anti-RSV antibody, or antigen-binding fragment thereof. In some embodiments, the anti-RSV antibody formulation comprises 175 mg/mL to 225 mg/mL of the anti-RSV antibody, or antigen-binding fragment thereof. In some embodiments, the anti-RSV antibody formulation comprises about 200 mg/ml of the anti-RSV antibody, or antigen-binding fragment thereof. In some embodiments, the anti-RSV antibody formulation comprises 200 mg/ml of the anti-RSV antibody, or antigen-binding fragment thereof.

In some embodiments of the above aspects and embodiments, the anti-RSV antibody formulation further comprises about 25 to about 75 μM diethylenetriamine pentaacetate (DTPA). In some embodiments, the anti-RSV antibody formulation further comprises 25 to 75 μM diethylenetriamine pentaacetate (DTPA).

In some embodiments of the above aspects and embodiments, the anti-RSV antibody formulation further comprises about 1 mM to about 20 mM L-methionine. In some embodiments, the anti-RSV antibody formulation further comprises 1 mM to 20 mM L-methionine.

In some embodiments, the pH of the anti-RSV antibody formulation is 5.5 to 6.5. In some embodiments, the pH of the anti-RSV antibody formulation is about 6.0. In some embodiments, the pH of the anti-RSV antibody formulation is 6.0.

In some embodiments, the anti-RSV antibody comprises CDRs having the amino acid sequences of SEQ ID NOs: 1-6. In some embodiments, the anti-RSV antibody comprises a heavy chain variable region having the amino acid sequence of SEQ ID NO: 7 and a light chain variable region having the amino acid sequence of SEQ ID NO: 8. In some embodiments, the anti-RSV antibody comprises a heavy chain immunoglobulin consisting of the amino acid sequence set forth in SEQ ID NO: 9 and a light chain immunoglobulin consisting of the amino acid sequence set forth in SEQ ID NO: 10.

In one aspect, the disclosure provides a method of treating or preventing RSV infection in a human patient in need thereof comprising: administering an effective amount of the anti-RSV antibody formulation of any one of the above aspects and embodiments to the patient.

In some embodiments of the foregoing method, the effective amount of the anti-RSV antibody formulation comprises a fixed dose of anti-RSV antibody or antigen-binding fragment thereof, of about 10 mg to about 150 mg. In some embodiments, the effective amount of the anti-RSV antibody formulation comprises a fixed dose of anti-RSV antibody or antigen-binding fragment thereof of 10 mg to 150 mg. In some embodiments, the effective amount of the anti-RSV antibody formulation comprises a fixed dose of anti-RSV antibody or antigen-binding fragment thereof between about 25 mg and about 125 mg. In some embodiments, the effective amount of the anti-RSV antibody formulation comprises a fixed dose of anti-RSV antibody or antigen-binding fragment thereof between 25 mg and 125 mg. In some embodiments, the effective amount of the anti-RSV antibody formulation comprises a fixed dose of anti-RSV antibody or antigen-binding fragment thereof between about 50 mg and about 100 mg. In some embodiments, the effective amount of the anti-RSV antibody formulation comprises a fixed dose of anti-RSV antibody or antigen-binding fragment thereof between 50 mg and 100 mg. In some embodiments, the effective amount of the anti-RSV antibody formulation comprises a fixed dose of anti-RSV antibody or antigen-binding fragment thereof of about 100 mg. In some embodiments, the effective amount of the anti-RSV antibody formulation comprises a fixed dose of anti-RSV antibody or antigen-binding fragment thereof of about 100 mg.

In some embodiments of the foregoing method, the anti-RSV antibody formulation is administered by intramuscular administration.

In some embodiments, the disclosure provides an anti-RSV antibody formulation of any one of the foregoing aspects or embodiments for use in a method for treating or preventing RSV infection in a human patient.

In some embodiments, the disclosure provides for use of the anti-RSV antibody formulation of any one of the foregoing aspects or embodiments in the manufacture of a medicament for treating or preventing RSV infection in a human patient.

In some embodiments of the invention, any of the formulations described herein is in an aqueous solution. In alternative embodiments, the invention provides lyophilized formulations made by lyophilizing an aqueous formulation to provide a reconstituted formulation of the invention, as discussed more fully, infra.

### VI. Lyophilized Pharmaceutical Compositions

Lyophilized formulations of therapeutic proteins (e.g., anti-RSV antibody, or an antigen-binding fragment thereof) provide several advantages. Lyophilized formulations in general offer better chemical stability than solution formulations, and thus increased half-life. A lyophilized formulation may also be reconstituted at different concentrations depending on clinical factors, such as route of administration or dosing. For example, a lyophilized formulation may be reconstituted at a high concentration (i.e. in a small volume) if necessary for subcutaneous administration, or at a lower concentration if administered intravenously. High concentrations may also be necessary if high dosing is required for a particular subject, particularly if administered subcutaneously where injection volume must be minimized. One such lyophilized antibody formulation is disclosed at U.S. Pat. No. 6,267,958, which is hereby incorporated by reference in its entirety. Lyophilized formulations of another therapeutic protein are disclosed at U.S. Pat. No. 7,247,707, which is hereby incorporated by reference in its entirety.

Typically, the lyophilized formulation is prepared in anticipation of reconstitution at high concentration of drug product (DP, in an exemplary embodiment an anti-RSV antibody, or antigen-binding fragment thereof), i.e. in anticipation of reconstitution in a low volume of water. Subsequent dilution with water or isotonic buffer can then readily be used to dilute the DP to a lower concentration. Typically, excipients are included in a lyophilized formulation of the invention at levels that will result in a roughly isotonic formulation when reconstituted at high DP concentration, e.g. for subcutaneous administration. Reconstitution in a larger volume of water to give a lower DP concentration will necessarily reduce the tonicity of the reconstituted solution, but such reduction may be of little significance in non-subcutaneous, e.g. intravenous, administration. If isotonicity is desired at lower DP concentration, the lyophilized powder may be reconstituted in the standard low volume of water and then further diluted with isotonic diluent, such as 0.9% sodium chloride.

In an embodiment of the invention, an anti-RSV antibody (or antigen-binding fragment thereof) is formulated as a lyophilized powder for reconstituting and utilizing for intravenous, subcutaneous, or intramuscular administration. In certain embodiments, the antibody (or antigen-binding fragment thereof) is provided at about 50 mg/vial, and is reconstituted with sterile water for injection prior to use. If desired, the reconstituted antibody may be aseptically diluted with 0.9% sodium chloride Injection USP in a sterile IV container. In some embodiments, the target pH of the reconstituted formulation is 5.5±0.5. In various embodiments, the lyophilized formulation of the invention enables reconstitution of the anti-RSV antibody to high concentrations, such as about 20, 25, 30, 40, 50, 60, 75, 100, 125, 150, 175 or more mg/mL.

Lyophilized formulations are by definition essentially dry, and thus the concept of concentration is not useful in describing them. Describing a lyophilized formulation in the terms of the weight of the components in a unit dose vial is more useful, but is problematic because it varies for different doses or vial sizes. In describing the lyophilized formulations of the invention, it is useful to express the amount of a component as the ratio of the weight of the component compared to the weight of the drug substance (DS) in the same sample (e.g. a vial). This ratio may be expressed as a percentage. Such ratios reflect an intrinsic property of the lyophilized formulations of the invention, independent of vial size, dosing, and reconstitution protocol.

In other embodiments, the lyophilized formulation of anti-RSV antibody, or antigen-binding fragment, is defined in terms of the pre-lyophilization solution used to make the lyophilized formulation, such as the pre-lyophilization solution. In one embodiment the pre-lyophilization solution comprises antibody, or antigen-binding fragment thereof, at a concentration of about 10 mg/mL, about 25 mg/mL, or about 50 mg/mL. Such pre-lyophilization solutions may be at pH 4.4-5.2 (including about 4.4, 4.5, 4.6, 4.7, 4.8, 4.9, 5.0, 5.1. and 5.2), e.g. preferably about pH 4.8, or about pH 5.5.

In yet other embodiments, the lyophilized formulation of anti-RSV antibody, or antigen-binding fragment, is defined in terms of the reconstituted solution generated from the lyophilized formulation.

Reconstituted solutions may comprise antibody, or antigen-binding fragment thereof, at concentrations of about 50, 60, 75, 80, 90 or 100 mg/mL or higher concentrations such as 150 mg/mL, 167 mg/mL, 200 mg/mL, or up to about 250 mg/mL. Such reconstituted solutions may be at about pH 5.5, or range from about pH 5.0 to about 6.0.

The lyophilized formulations of the invention are formed by lyophilization (freeze-drying) of a pre-lyophilization solution. Freeze-drying is accomplished by freezing the formulation and subsequently subliming water at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will dictate the time required for drying, which can range from a few hours to several days (e.g. 40-60 hrs). A secondary drying stage may be carried out at about 0-40° C., depending primarily on the type and size of container and the type of protein employed. The secondary drying time is dictated by the desired residual moisture level in the product and typically takes at least about 5 hours. Typically, the moisture content of a lyophilized formulation is less than about 5%, and preferably less than about 3%. The pressure may be the same as that employed during the primary drying step. Freeze-drying conditions can be varied depending on the formulation and vial size.

In some instances, it may be desirable to lyophilize the protein formulation in the container in which reconstitution of the protein is to be carried out in order to avoid a transfer step. The container in this instance may, for example, be a 3, 5, 10, 20, 50 or 100 cc vial.

The lyophilized formulations of the invention are reconstituted prior to administration. The protein may be reconstituted at a concentration of about 10, 15, 20, 25, 30, 40, 50, 60, 75, 80, 90 or 100 mg/mL or higher concentrations such as 150 mg/mL, 200 mg/mL, 250 mg/mL, or 300 mg/mL up to about 500 mg/mL. High protein concentrations are particularly useful where subcutaneous delivery of the reconstituted formulation is intended. However, for other routes of administration, such as intravenous administration, lower concentrations of the protein may be desired (e.g. from about 5-50 mg/mL).

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWFI), a pH buffered solution (e.g. phosphate-buffered saline), sterile saline solution, Ringer's solution or dextrose solution.

The disclosure provides a liquid anti-RSV antibody formulation that is reconstituted from a lyophilized formulation wherein the reconstituted solution comprises: a) about 50 mg/mL to about 150 mg/mL of an anti-RSV antibody, or antigen-binding fragment thereof, b) about 5 mM to about 20 mM histidine; c) about 6% to about 8% weight/volume (w/v) sucrose; d) an excipient selected from about 25 mM to about 75 mM of L-arginine or a pharmaceutically acceptable salt of L-arginine, about 25 mM to about 75 mM of L-proline or a pharmaceutically acceptable salt of L-proline, about 25 mM to about 75 mM of L-glutamate or a pharmaceutically acceptable salt of L-glutamate, about 25 mM to about 75 mM L-lysine or a pharmaceutically acceptable salt of L-lysine, about 25 mM to about 75 mM of a mixture of L-arginine and L-glutamate, and about 25 mM to about 75 mM of a mixture of L-arginine(HCl) and L-lysine(HCl); e) about 25 μM to 75 μM DTPA; f) about 0.01% to about 0.10% polysorbate 80; and optionally g) about 1 mM to about 20 mM L-methionine.

The disclosure provides a liquid anti-RSV antibody formulation that is reconstituted from a lyophilized formulation wherein the reconstituted solution comprises: a) 100 mg/mL of an anti-RSV antibody, or antigen-binding fragment thereof, b) 10 mM histidine; c) 7% weight/volume (w/v) sucrose; d) an excipient selected from 50 mM L-arginine(HCl), 50 mM L-lysine(HCl), a mixture of 25 mM L-arginine and 25 mM L-glutamate, and a mixture of 25 mM L-arginine(HCl) and 25 mM L-lysine(HCl); e) 50 μM DTPA; f) 0.02% polysorbate 80; and optionally g) 1 mM to 20 mM L-methionine.

The disclosure provides a liquid anti-RSV antibody formulation that is reconstituted from a lyophilized formulation wherein the reconstituted solution comprises: a) 100 mg/mL of an anti-RSV antibody, or antigen-binding fragment thereof, b) 10 mM histidine; c) 7% weight/volume (w/v) sucrose; d) a mixture of 25 mM L-arginine(HCl) and 25 mM L-lysine(HCl); e) 50 μM DTPA; and f) 0.02% polysorbate 80.

The disclosure also provides a liquid anti-RSV antibody formulation that is reconstituted from a lyophilized formulation wherein the reconstituted solution comprises: a) about 50 mg/mL to about 250 mg/mL of an anti-RSV antibody, or antigen-binding fragment thereof, b) about 5 mM to about 20 mM histidine; c) about 4% to about 8% (w/v) sucrose; d) an excipient selected from the group consisting of about 25 mM to about 75 mM of L-arginine or a pharmaceutically acceptable salt of L-arginine, about 25 mM to about 75 mM of L-lysine or a pharmaceutically acceptable salt of L-lysine, a mixture of about 25 mM to about 75 mM L-arginine or a pharmaceutically acceptable salt of L-arginine and about 25 mM to about 75 mM L-glutamate or a pharmaceutically acceptable salt of L-glutamate, and a mixture of about 25 mM to about 75 mM of L-arginine or a pharmaceutically acceptable salt of L-arginine and about 25 mM to about 75 mM L-lysine or a pharmaceutically acceptable salt of L-lysine; and e) about 0.01% to about 0.10% (w/v) polysorbate 80.

### VII. Liquid Pharmaceutical Compositions

A liquid antibody formulation can be made by taking the drug substance (e.g., anti-RSV antibody) which is in liquid form and buffer exchanging it into the desired buffer as the last step of the purification process. There is no lyophilization step in this embodiment. The drug substance in the final buffer is concentrated to a desired concentration. Excipients such as sucrose, methionine and polysorbate 80 are added to the drug substance and it is diluted using the appropriate buffer to final protein concentration. The final formulated drug substance is filtered, e.g. using 0.22 m filters, and filled into a final container (e.g. glass vials or syringes). Such a liquid formulation is exemplified by a final liquid formulation comprising a) 100 mg/mL of an anti-RSV antibody, or antigen-binding fragment thereof, b) 10 mM histidine; c) 7% weight/volume (w/v) sucrose; d) a mixture of 25 mM L-arginine(HCl) and 25 mM L-lysine(HCl); e) 50 μM DTPA; and f) 0.02% polysorbate 80.

### VIII. Methods of Use

The invention also relates to a method of preventing or treating RSV infection in a subject, the method comprising administering an effective amount of any of the formulations of the invention; i.e., any formulation described herein, to the subject. In some embodiments of this method, the formulation is administered to the subject via intravenous administration. In other embodiments, the formulation is administered to the subject via subcutaneous administration. In other embodiments, the formulation is administered via intramuscular administration.

In a specific embodiment, a mammal, preferably a human, is administered a prophylactic, therapeutic or pharmaceutical formulation of the present invention for the treatment, prevention or amelioration of one or more symptoms associated with a RSV infection in an amount effective for decreasing RSV titers. In accordance with this embodiment, an effective amount of an anti-RSV antibody or antibody fragment reduces the RSV titers in the lung as measured, for example, by the concentration of RSV in sputum samples or a lavage from the lungs from a mammal. In another embodiment, a mammal, preferably a human, is administered a prophylactic, therapeutic or pharmaceutical formulation comprising an anti-RSV antibody of the present invention or fragments thereof for the treatment, prevention or amelioration of symptoms associated with a RSV infection in an amount effective for neutralizing RSV and/or blocking RSV infection in the mammal.

The formulations of the instant invention can also be used immunotherapeutically for RSV disease in both humans and other animals. The term, “immunotherapeutically” or “immunotherapy” as used herein in conjunction with the anti-RSV antibody or antigen-binding fragments thereof of the invention denotes both prophylactic as well as therapeutic administration and both passive immunization with substantially purified polypeptide products, as well as gene therapy by transfer of polynucleotide sequences encoding the product or part thereof. Passive immunization includes transfer of active humoral immunity or providing antibodies to a subject in need thereof. Accordingly, in certain embodiments of the invention, the present invention provides methods for transfer of active humoral immunity and methods of providing RSV antibodies or antigen-binding fragments thereof, such as IgG antibodies, to a patient at risk of RSV infection. Thus, the monoclonal antibodies or antigen-binding fragments thereof can be administered to high-risk subjects in order to lessen the likelihood and/or severity of RSV disease or administered to subjects already evidencing active RSV infection.

The present invention also provides a method for modulating or treating at least one adult or pediatric RSV related disease, in a cell, tissue, organ, animal, or patient including, but not limited to, lower respiratory infections, pneumonia, tracheobronchitis, bronchiolitis, bronchitis, and any related infections or inflammatory disorders, such as but not limited to at least one of, or at least one inflammation related to, systemic inflammatory response syndrome, sepsis syndrome, gram positive sepsis, gram negative sepsis, culture negative sepsis, fungal sepsis, neutropenic fever, urosepsis, meningococcemia, adult respiratory distress syndrome, allergic rhinitis, perennial rhinitis, asthma, systemic anaphylaxis, receptor hypersensitivity reactions, chronic obstructive pulmonary disease (COPD), hypersensitivity pneumonitis, granulomas due to intracellular organisms, drug sensitivity, cachexia, cystic fibrosis, neonatal chronic lung disease; at least one infectious disease in a cell, tissue, organ, animal or patient, including, but not limited to, at least one of: acute or chronic bacterial infection, acute and chronic parasitic or infectious processes, including bacterial, viral and fungal infections, HIV infection, HIV neuropathy, meningitis, hepatitis (A,B or C, or the like), septic arthritis, peritonitis, pneumonia, epiglottitis, E. coli 0157:h7, hemolytic uremic syndrome, thrombolytic thrombocytopenic purpura, malaria, dengue hemorrhagic fever, leishmaniasis, leprosy, toxic shock syndrome, streptococcal myositis, gas gangrene, Mycobacterium tuberculosis, Mycobacterium avium intracellulare, Pneumocystis carinii pneumonia, pelvic inflammatory disease, orchitis, epididymitis, legionella, lyme disease, influenza A, Epstein-Barr virus, vital-associated hemophagocytic syndrome, vital encephalitis, aseptic meningitis, and the like. Such a method can optionally comprise administering an effective amount of a composition or pharmaceutical composition comprising at least one RSV antibody or antigenic fragment thereof to a cell, tissue, organ, animal or patient in need of such modulation, treatment or therapy.

In one embodiment, prophylactic, therapeutic or pharmaceutical formulation comprising an anti-RSV antibody of the invention or fragments thereof are administered to a mammal, preferably a human, to treat, prevent or ameliorate one or more symptoms associated with RSV infection. In another embodiment, prophylactic, therapeutic or pharmaceutical formulations comprising an anti-RSV antibody of the invention or fragments thereof are administered to a human with cystic fibrosis, bronchopulmonary dysplasia, congenital heart disease, congenital immunodeficiency or acquired immunodeficiency, or to a human who has had a transplant (e.g., bone marrow, lung, or hematopoietic stem cell transplantation (HSCT)) to treat, prevent or ameliorate one or more symptoms associated with RSV infection.

In another embodiment, prophylactic, therapeutic or pharmaceutical formulations comprising an anti-RSV antibody of the invention or fragments thereof are administered to a human infant, preferably a human infant born prematurely or a human infant at risk of hospitalization for RSV infection to treat, prevent or ameliorate one or more symptoms associated with RSV infection. In yet another embodiment, prophylactic, therapeutic or pharmaceutical formulations of the instant invention are administered to the elderly or people in group homes (e.g., nursing homes or rehabilitation centers) or immunocompromised individuals.

In another embodiment, the present invention provides a method of preventing respiratory infection caused by RSV. In another embodiment, the present invention provides a method of preventing lower respiratory infection caused by RSV. In another embodiment, the present invention provides a method of preventing lower respiratory infection caused by RSV A and B strains. In another embodiment, the present invention provides a method of preventing medically attended lower respiratory infection caused by RSV A and B strains in infants born >29 weeks of gestation and <8 months of age at time of dosing. In another embodiment, the present invention provides a method of preventing lower respiratory tract infection caused by RSV in all infants entering their first RSV season and children with chronic lung disease or congenital heart disease entering their first and second RSV season.

### IX. General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausubel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI Suite (Informax, Inc, Bethesda, MD); GCG Wisconsin Package (Accelrys, Inc., San Diego, CA); DECYPHER (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### X. Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and FTIR. SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### Example 1

### Anti-RSV Antibody Formulation Stability at Different pH

This study was performed with a 100 mg/mL anti-RSV antibody concentration in 10 mM histidine buffer. Seven % (w/v) sucrose was added to the formulation to increase the bulk stability (as stabilizer and nonionic tonicity modifier) of the molecule. Briefly, the drug substance (DS) was formulated at 100 mg/mL in 10 mM histidine, 7% sucrose at pH 5.5, pH 6.0 and pH 6.5. Formulated DS was filled into vials and staged on stability at 5° C. (ambient humidity), 25° C. (60% relative humidity), and 40° C. (75% relative humidity) for three months protected from light.

Samples were assessed by size exclusion chromatography (SEC) for purity in which the percentage of monomer was determined, as well as the percentages of high molecular weight species (HMW) and late eluting peaks (LMW species). Ultra Performance—Size Exclusion Chromatography (UP-SEC) was performed by diluting the samples (anti-RSV antibody) to 1.0 mg/mL in mobile phase (100 mM sodium phosphate and 100 mM sodium chloride, pH 7.0); flow rate of 0.5 mL/min. The diluted samples were injected (5 μL) into a UPLC equipped with a Waters BEH200 column and a UV detector. Proteins in the sample were separated by size and detected by UV absorption at 214 nm.

This study was conducted to study the effect of pH on stability of an anti-RSV antibody formulation. Based on the results from the thermal stability study for up to 3 months, there was no measurable difference between the stability of 100 mg/mL formulations at pH 5.5, 6.0, and 6.5 for any of the conditions. No change was noticed in UP-SEC % Monomer levels at 5° C.; however, a slight drop (~2%) was noticed at 25° C. and a more significant drop (~20%) at 40° C. Based on the results, a pH of 6.0 was selected for further development (see Table 1).

TABLE 1

| Formulation | | | | | |
|---|---|---|---|---|---|
| Number | Description | | | | |
| A1 | Anti-RSV antibody | 10 mM | 7% Sucrose | 0.02% PS80 | pH = 5.5 |
| | (100 mg/mL) | Histidine | (w/v) | (w/v) | |
| A2 | Anti-RSV antibody | 10 mM | 7% Sucrose | 0.02% PS80 | pH = 6.0 |
| | (100 mg/mL) | Histidine | (w/v) | (w/v) | |
| A3 | Anti-RSV antibody | 10 mM | 7% Sucrose | 0.02% PS80 | pH = 6.5 |
| | (100 mg/mL) | Histidine | (w/v) | (w/v) | |

### Example 2

### Anti-RSV Antibody Formulation Excipient (Amino Acids and Mixtures Thereof) Screening (to Reduce Aggregation)

This study compared the stability of an anti-RSV antibody in 10 mM histidine (pH=6), 0.02% PS80, 7% sucrose in the presence of (i) 50 mM L-Arg.HCl or (ii) 50 mM L-Lys.HCl or (iii) mixture of 25 mM L-Arg and 25 mM L-Glu or (iv) mixture of 25 mM L-Arg.HCl and 25 mM L-Lys.HCl.

These four excipients were tested to reduce aggregation, improve chemical stability (see Example 3) and reduce viscosity (see Example 4). The four tested excipients were:
(i) 50 mM L-Arg.HCl;
(ii) 50 mM L-Lys.HCl;
(iii) a mixture of 25 mM L-Arg and 25 mM L-Glu; and
(iv) a mixture of 25 mM L-Arg.HCl and 25 mM L-Lys.HCl.

The first two excipients were examined to understand the effects of the positively charged lysine and arginine in the presence of the chloride counteranion. The third excipient was tested to understand the effect of arginine in the presence of another counteranion (glutamate). The fourth excipient was a mixture of the first two excipients to determine if the two excipients can play a complementary role. It was hypothesized that this mixture may play a better role than the individual excipients, as the mixture of excipients have different pKa and differentially shield the surface change distribution of the mAb, thereby increasing repulsive interactions, which in turn would reduce aggregation and viscosity.

To evaluate the stability of the formulations, the five formulations were filled into vials and staged on stability at 5° C. (ambient humidity), 25′ C (60% relative humidity), and 40° C. (75% relative humidity) for eight weeks protected from light. The five formulations are listed in Table 2 below.

TABLE 2

| Formulation | | | | | |
|---|---|---|---|---|---|
| Number | Description | | | | |
| 1 | Anti-RSV antibody | 10 mM | 7% Sucrose | 0.02% PS80 | N/A |
| | (100 mg/mL) | Histidine | (w/v) | (w/v) | |
| | | (pH = 6) | | | |
| 2 | Anti-RSV antibody | 10 mM | 7% Sucrose | 0.02% PS80 | 50 mM L- |
| | (100 mg/mL) | Histidine | (w/v) | (w/v) | Arg•HCl |
| | | (pH = 6) | | | |
| 3 | Anti-RSV antibody | 10 mM | 7% Sucrose | 0.02% PS80 | 50 mM L- |
| | (100 mg/mL) | Histidine | (w/v) | (w/v) | Lys•HCl |
| | | (pH = 6) | | | |
| 4 | Anti-RSV antibody | 10 mM | 7% Sucrose | 0.02% PS80 | Mixture of 25 mM |
| | (100 mg/mL) | Histidine | (w/v) | (w/v) | L-Arg and 25 mM |
| | | (pH = 6) | | | L-Glu |
| 5 | Anti-RSV antibody | 10 mM | 7% Sucrose | 0.02% PS80 | Mixture of 25 mM |
| | (100 mg/mL) | Histidine | (w/v) | (w/v) | L-Arg•HCl and 25 |
| | | (pH = 6) | | | mM L-Lys•HCl |

Samples were assessed by size exclusion chromatography (SEC) for purity in which the percentage of monomer was determined, as well as the percentages of high molecular weight species (HMW) and late eluting peaks (LMW species). Ultra Performance—Size Exclusion Chromatography (UP-SEC) was performed by diluting the samples to 1.0 mg/mL (anti-RSV antibody) in mobile phase (100 mM sodium phosphate and 100 mM sodium chloride, pH 7.0); flow rate of 0.5 mL/min. The diluted samples were injected (5 μL) into a UPLC equipped with a Waters BEH200 column and a UV detector. Proteins in the sample were separated by size and detected by UV absorption at 214 nm.

The UPSEC data to evaluate the levels of High Molecular Weight Species (HMW or aggregates), 00 monomer and Low Molecular Weight (LMW) species is shown in the Table 3. [T0=Time 0; 4W=4 Weeks; and 8W=8 Weeks]

TABLE 3

| | Peak | | |
|---|---|---|---|
| Sample Name | HMW | Monomer | LMW |
| Anti-RSV Antibody DP control T0 5° C. | 1.77 | 98.1 | 0.09 |
| Anti-RSV Antibody DP control T0 4W5° C. | 1.88 | 98.0 | 0.11 |
| Anti-RSV Antibody DP control T0 4W25° C. | 2.86 | 96.9 | 0.21 |
| Anti-RSV Antibody DP control T0 4W40° C. | 4.05 | 93.2 | 2.77 |
| Anti-RSV Antibody DP control T0 8W5° C. | 2.00 | 97.9 | 0.13 |
| Anti-RSV Antibody DP control T0 8W25° C. | 3.13 | 96.6 | 0.28 |
| Anti-RSV Antibody DP control T0 8W40° C. | 4.83 | 91.1 | 4.05 |
| Anti-RSV Antibody DP + L-Arg•HCl T05° C. | 1.55 | 98.3 | 0.14 |
| Anti-RSV Antibody DP + L-Arg•HCl 4W5° C. | 1.54 | 98.3 | 0.12 |
| Anti-RSV Antibody DP + L-Arg•HCl 4W25° C. | 2.06 | 97.8 | 0.18 |
| Anti-RSV Antibody DP + L-Arg•HCl 4W40° C. | 2.72 | 95.4 | 1.90 |
| Anti-RSV Antibody DP + L-Arg•HCl 8W5° C. | 1.66 | 98.2 | 0.13 |
| Anti-RSV Antibody DP + L-Arg•HCl 8W25° C. | 2.34 | 97.4 | 0.28 |
| Anti-RSV Antibody DP + L-Arg•HCl 8W40° C. | 3.46 | 93.1 | 3.41 |
| Anti-RSV Antibody DP + L-Lys•HCl T05° C. | 1.58 | 98.3 | 0.12 |
| Anti-RSV Antibody DP + L-Lys•HCl 4W5° C. | 1.79 | 98.1 | 0.12 |
| Anti-RSV Antibody DP + L-Lys•HCl 4W25° C. | 2.17 | 97.6 | 0.20 |
| Anti-RSV Antibody DP + L-Lys•HCl 4W40° C. | 3.01 | 95.1 | 1.87 |
| Anti-RSV Antibody DP + L-Lys•HCl 8W5° C. | 1.71 | 98.2 | 0.13 |
| Anti-RSV Antibody DP + L-Lys•HCl 8W25° C. | 2.51 | 97.2 | 0.27 |
| Anti-RSV Antibody DP + L-Lys•HCl 8W40° C. | 3.94 | 92.5 | 3.58 |
| Anti-RSV Antibody DP + L-Arg•L-Glu T05° C. | 1.80 | 98.1 | 0.10 |
| Anti-RSV Antibody DP + L-Arg•L-Glu 4W5° C. | 1.87 | 98.0 | 0.12 |
| Anti-RSV Antibody DP + L-Arg•L-Glu 4W25° C. | 2.49 | 97.3 | 0.22 |
| Anti-RSV Antibody DP + L-Arg•L-Glu 4W40° C. | 3.77 | 93.4 | 2.83 |
| Anti-RSV Antibody DP + L-Arg•L-Glu 8W5° C. | 1.90 | 98.0 | 0.13 |
| Anti-RSV Antibody DP + L-Arg•L-Glu 8W25° C. | 2.68 | 97.0 | 0.31 |
| Anti-RSV Antibody DP + L-Arg•L-Glu 8W40° C. | 4.40 | 91.8 | 3.80 |
| Anti-RSV Antibody DP + L-Arg•HCl + L-Lys•HCl T05° C. | 1.67 | 98.2 | 0.10 |
| Anti-RSV Antibody DP + L-Arg•HCl + L-Lys•HCl 4W5° C. | 2.22 | 97.6 | 0.20 |
| Anti-RSV Antibody DP + L-Arg•HCl + L-Lys•HCl 4W25° C. | 1.79 | 98.1 | 0.12 |
| Anti-RSV Antibody DP + L-Arg•HCl + L-Lys•HCl 4W40° C. | 3.03 | 94.8 | 2.19 |
| Anti-RSV Antibody DP + L-Arg•HCl + L-Lys•HCl 8W5° C. | 1.71 | 98.2 | 0.13 |
| Anti-RSV Antibody DP + L-Arg•HCl + L-Lys•HCl 8W25° C. | 2.34 | 97.4 | 0.25 |
| Anti-RSV Antibody DP + L-Arg•HCl + L-Lys•HCl 8W40° C. | 3.36 | 93.7 | 2.95 |

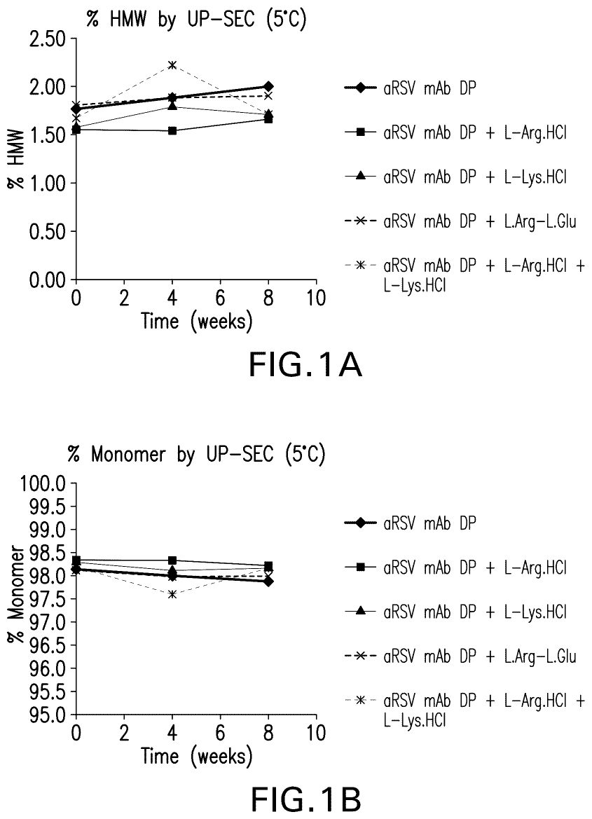

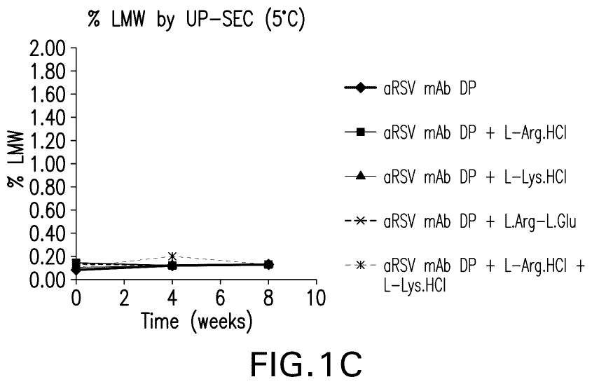

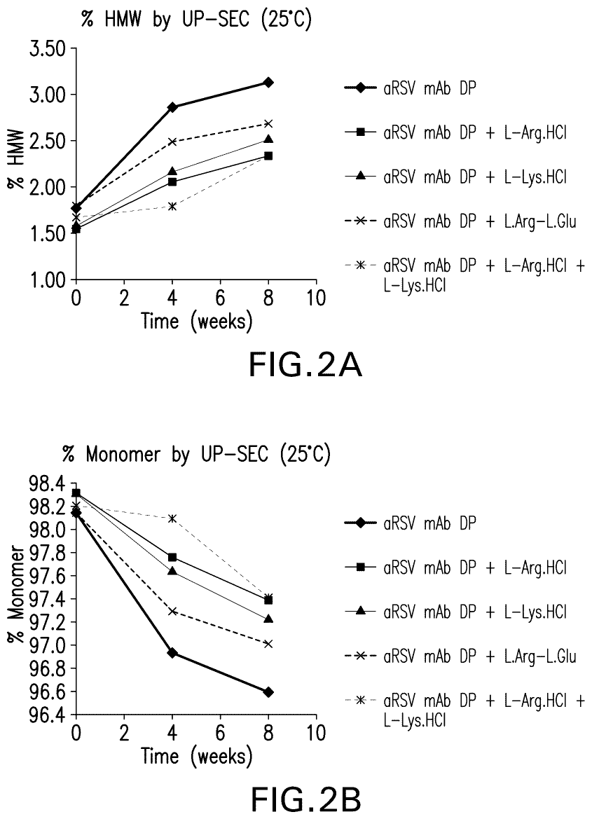

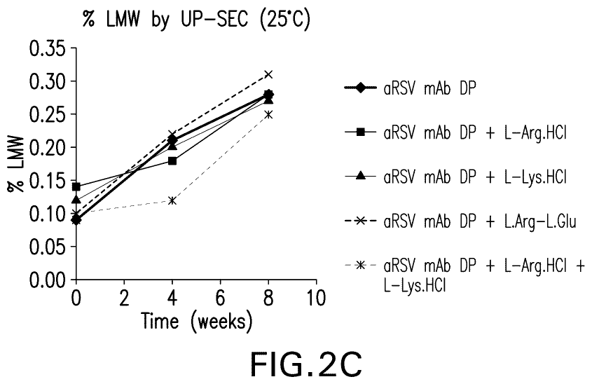

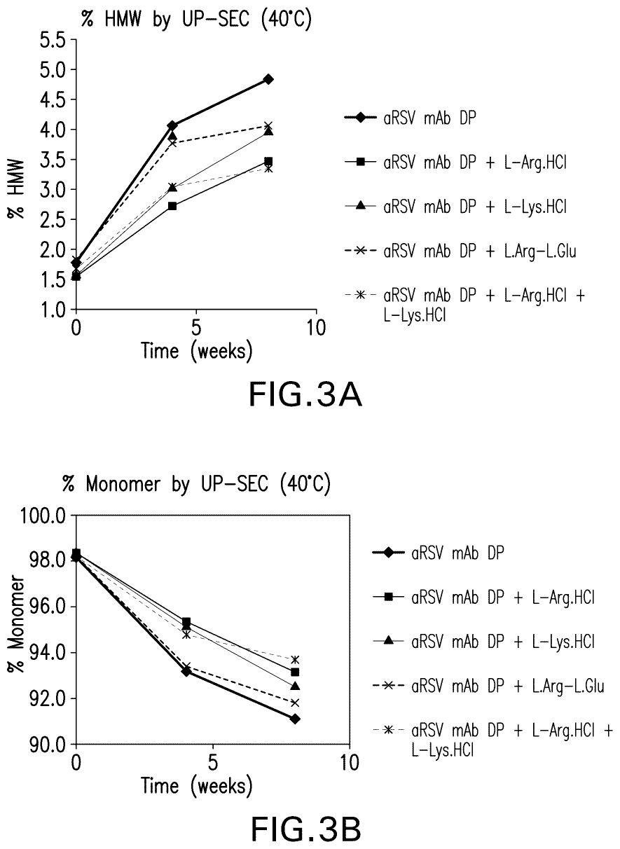

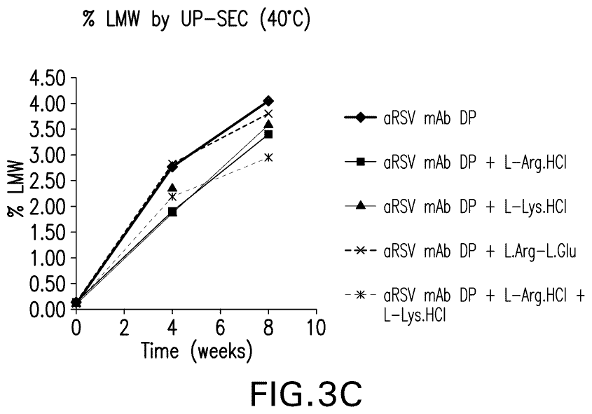

As shown in FIG. 1, FIG. 2, and FIG. 3, UP-SEC analysis of the samples to determine the percentage of HMW and percentage of monomer indicated that at 5° C., 25° C. and 40° C., all the formulations showed a trend of increase in % HMW peak and % LMW peak (and a consequent decrease in % monomer peak) up to the 8-week time point. At 25° C., all the formulations showed similar trends, but smaller changes, as compared to 40° C. At 5° C., no substantial changes were observed.

As shown in FIG. 2 and FIG. 3, formulation 1 shows a greater increase in % HMW and % LMW as compared to the other formulations. Additionally, formulation 1 showed a greater decrease of % monomer. Thus, all four sets of excipients decrease the % HMW and % LMW and thus improve the stability of the anti-RSV antibody DP.

### Example 3

### Anti-RSV Antibody Formulation Excipient Screening (Amino Acids and Mixtures Thereof) to Improve Chemical Stability of the Anti-RSV Antibody

This study compared the stability of an anti-RSV antibody in 10 mM histidine (pH=6), 0.02% PS80, 7% sucrose in the presence of (i) 50 mM L-Arg.HCl or (ii) 50 mM L-Lys.HCl or (iii) mixture of 25 mM L-Arg and 25 mM L-Glu or (iv) mixture of 25 mM L-Arg.HCl and 25 mM Lys.HCl.

These four excipients were tested to improve chemical stability. The four tested excipients were:
(i) 50 mM L-Arg.HCl;
(ii) 50 mM L-Lys.HCl;
(iii) a mixture of 25 mM L-Arg and 25 mM L-Glu; and
(iv) a mixture of 25 mM L-Arg.HCl and 25 mM Lys.HCl.

The first two excipients were examined to understand the effects of the positively charged lysine and arginine in the presence of the chloride counteranion. The third excipient was tested to understand the effect of arginine in the presence of another counteranion (glutamate). The fourth excipient was a mixture of the first two excipients to determine whether the two excipients can play a complementary role. It was hypothesized that this mixture may play a better role than the individual excipients, as the mixture of excipients have different pKa and differentially shields the surface change distribution of the mAb, and this may impact chemical degradation of the mAb.

To evaluate the stability of the formulations, the five formulations were filled into vials and staged on stability at 5° C. (ambient humidity), 25° C. (60% relative humidity), and 40° C. (75% relative humidity) for eight weeks protected from light. The five formulations are as shown in Example 2.

Ion exchange chromatography was performed to evaluate the chemical stability and to monitor the change in the charge variant profile over time. An ion exchange HPLC method was performed using a Dionex ProPac WCX-10 column and a UV detector at 280 nm. Samples were diluted in purified water, and 80 μg were injected for analysis. The mobile phase used for the IEX analysis of the thermal stability samples was a gradient of the following mobile phases (mobile phase A:20 mM MOPS, pH 7.2; mobile phase B: 50 mM sodium phosphate, 60 mM sodium chloride pH 8.0). The assay is performed using a mobile phase gradient from 20 mM MOPS, pH 7.2 to 50 mM sodium phosphate, 60 mM NaCl, pH 8.0. UV detection was performed at 280 nm.

The HP-IEX data to evaluate the levels of Acidic Variants, % Main Peak and % Basic Variants is shown in Table 4. [T0=Time 0; 4W=4 Weeks; and 8W=8 Weeks]

TABLE 4

| | Reportable % Peak Areas | | |
|---|---|---|---|
| | Acidic | Total | Basic |
| Sample Name | Variants | Main | Variants |
| Anti-RSV Antibody DP control T05° C. | 25.18 | 58.1 | 16.68 |
| Anti-RSV Antibody DP control T05° C. 4W5° C. | 24.97 | 58.9 | 16.10 |
| Anti-RSV Antibody DP control T05° C. 4W25° C. | 28.41 | 54.1 | 17.47 |
| Anti-RSV Antibody DP control T05° C. 4W40° C. | 44.06 | 37.5 | 18.41 |
| Anti-RSV Antibody DP control T05° C. 8W5° C. | 24.57 | 59.0 | 16.38 |
| Anti-RSV Antibody DP control T05° C. 8W25° C. | 30.62 | 52.0 | 17.38 |
| Anti-RSV Antibody DP control T05° C. 8W40° C. | 53.49 | 28.7 | 17.82 |
| Anti-RSV Antibody DP + L-Arg•HCl T05° C. | 24.23 | 59.8 | 15.92 |
| Anti-RSV Antibody DP + L-Arg•HCl 4W5° C. | 24.43 | 59.2 | 16.35 |
| Anti-RSV Antibody DP + L-Arg•HCl 4W25° C. | 26.05 | 56.6 | 17.39 |
| Anti-RSV Antibody DP + L-Arg•HCl 4W40° C. | 35.13 | 46.0 | 18.88 |
| Anti-RSV Antibody DP + L-Arg•HCl 8W5° C. | 24.00 | 59.7 | 16.34 |
| Anti-RSV Antibody DP + L-Arg•HCl 8W25° C. | 28.25 | 53.2 | 18.54 |
| Anti-RSV Antibody DP + L-Arg•HCl 8W40° C. | 48.43 | 32.6 | 18.96 |
| Anti-RSV Antibody DP + Lys•HCl T05° C. | 23.98 | 60.3 | 15.72 |
| Anti-RSV Antibody DP + Lys•HCl 4W5° C. | 24.08 | 59.8 | 16.09 |
| Anti-RSV Antibody DP + Lys•HCl 4W25° C. | 25.25 | 57.4 | 17.37 |
| Anti-RSV Antibody DP + Lys•HCl 4W40° C. | 34.94 | 45.7 | 19.36 |
| Anti-RSV Antibody DP + Lys•HCl 8W5° C. | 23.95 | 59.9 | 16.14 |
| Anti-RSV Antibody DP + Lys•HCl 8W25° C. | 27.79 | 53.6 | 18.59 |
| Anti-RSV Antibody DP + Lys•HCl 8W40° C. | 48.05 | 32.7 | 19.28 |
| Anti-RSV Antibody DP + L-Arg•L-Glu T05° C. | 24.55 | 59.5 | 15.92 |
| Anti-RSV Antibody DP + L-Arg•L-Glu 4W5° C. | 24.34 | 59.7 | 15.94 |
| Anti-RSV Antibody DP + L-Arg•L-Glu 4W25° C. | 29.80 | 52.6 | 17.63 |
| Anti-RSV Antibody DP + L-Arg•L-Glu 4W40° C. | 47.35 | 34.8 | 17.89 |
| Anti-RSV Antibody DP + L-Arg•L-Glu 8W5° C. | 24.46 | 59.4 | 16.14 |
| Anti-RSV Antibody DP + L-Arg•L-Glu 8W25° C. | 32.22 | 49.2 | 18.55 |
| Anti-RSV Antibody DP + L-Arg•L-Glu 8W40° C. | 55.41 | 26.8 | 17.77 |
| Anti-RSV Antibody DP + L-Arg•HCl + Lys•HCl T05° C. | 23.40 | 60.9 | 15.68 |
| Anti-RSV Antibody DP + L-Arg•HCl + Lys•HCl 4W5° C. | 22.84 | 61.2 | 15.93 |
| Anti-RSV Antibody DP + L-Arg•HCl + Lys•HCl 4W25° C. | 25.02 | 57.5 | 17.45 |
| Anti-RSV Antibody DP + L-Arg•HCl + Lys•HCl 4W40° C. | 36.75 | 44.0 | 19.29 |
| Anti-RSV Antibody DP + L-Arg•HCl + Lys•HCl 8W5° C. | 22.71 | 61.4 | 15.93 |
| Anti-RSV Antibody DP + L-Arg•HCl + Lys•HCl 8W25° C. | 25.97 | 56.1 | 17.97 |
| Anti-RSV Antibody DP + L-Arg•HCl + Lys•HCl 8W40° C. | 42.05 | 38.9 | 19.04 |

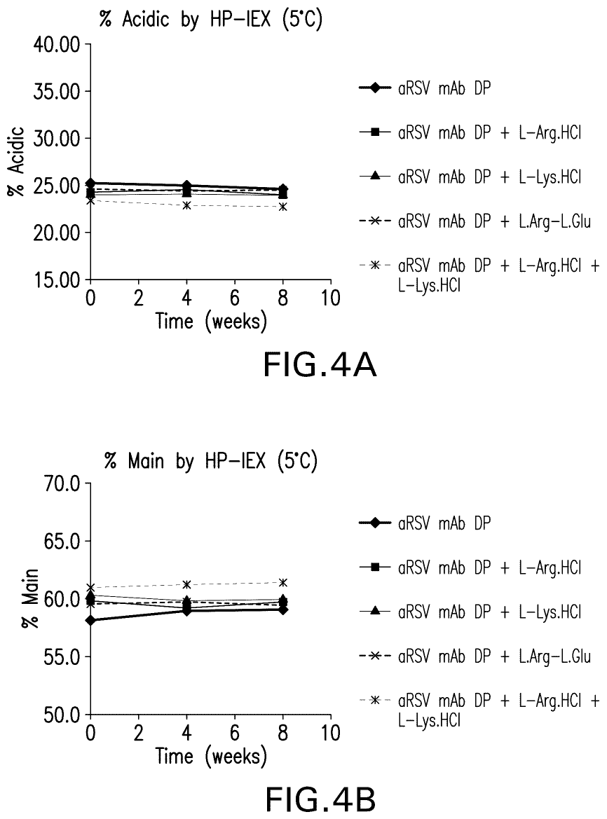

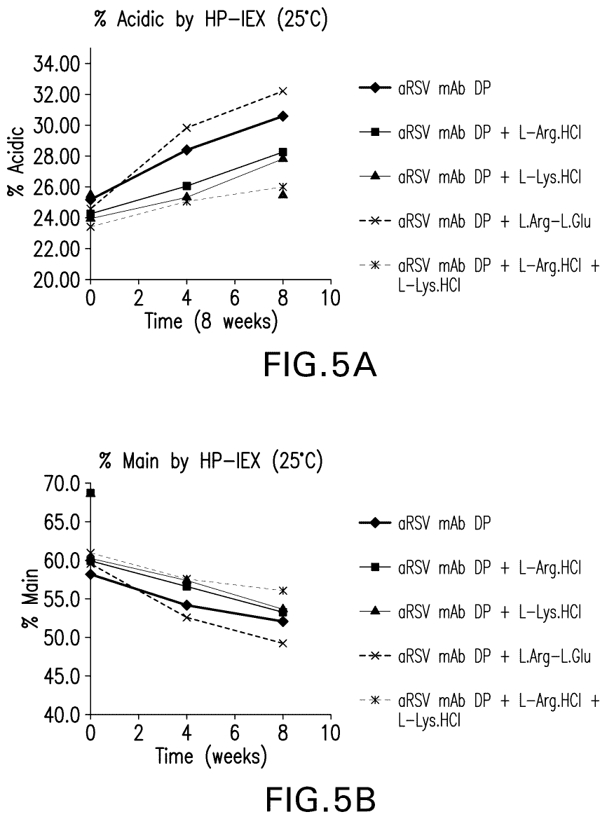

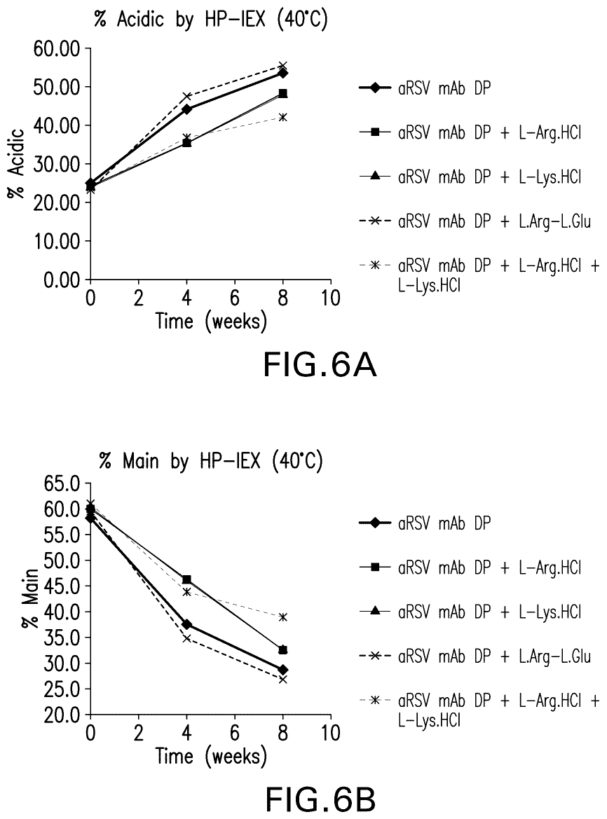

As shown in FIG. 4, FIG. 5 and FIG. 6, HP-IEX analysis of the samples to determine the chemical stability indicated that at 5° C., 25° C. and 40° C., all the formulations showed a trend towards an increase in % acidic peak and a consequent decrease in % monomer peak up to the 8-week time point. At 25° C., all the formulations showed similar trends, but smaller changes, as compared to 40° C. At 5° C., no substantial changes were observed.

As seen in FIG. 5 and FIG. 6, formulation 1 shows a greater increase in % acidic peak as compared to the other formulations. Additionally, formulation 1 showed a greater decrease of % main peak as compared to the other formulations. Thus, all the four sets of excipients decrease the % acidic peak and thus improves the chemical stability of the anti-RSV antibody DP.

### Example 4

### Anti-RSV Antibody Formulation Excipient Screening (to Decrease Viscosity of the Anti-RSV Antibody)

This study compared the stability of an anti-RSV antibody in 10 mM histidine (pH=6), 0.02% PS80, 7% sucrose in the presence of (i) 50 mM L-Arg.HCl or (ii) 50 mM L-Lys.HCl or (iii) mixture of 25 mM L-Arg and 25 mM L-Glu or (iv) mixture of 25 mM L-Arg.HCl and 25 mM L-Lys.HCl.

These four excipients were tested for their ability to decrease viscosity of the anti-RSV antibody. The four tested excipients were:
(i) 50 mM L-Arg.HCl;
(ii) 50 mM L-Lys.HCl;
(iii) a mixture of 25 mM L-Arg and 25 mM L-Glu; and
(iv) a mixture of 25 mM L-Arg.HCl and 25 mM L-Lys.HCl.

The first two excipients were examined to understand the effects of the positively charged lysine and arginine in the presence of the chloride counteranion. The third excipient was tested to understand the effect of arginine in the presence of another counteranion (glutamate). The fourth excipient was a mixture of the first two excipients to see if the two excipients can play a complementary role. It was hypothesized that this mixture may play a better role than the individual excipients, as the mixture of excipients have different pKa and differentially shields the surface change distribution of the mAb, thereby increasing the repulsive protein-protein interaction and decreasing viscosity of the mAb.

To measure viscosity, the samples were loaded into a 500 μl syringe and viscosity was measured (five times for each sample) at 20° C. with an MVROC Viscometer. VROC sensors detected viscosity by measuring the pressure drop as samples flow through the sensor's flow channel at designated positions from the inlet. Viscosity was measured as a function of shear rate with shear rates of samples being determined by sample viscosity.

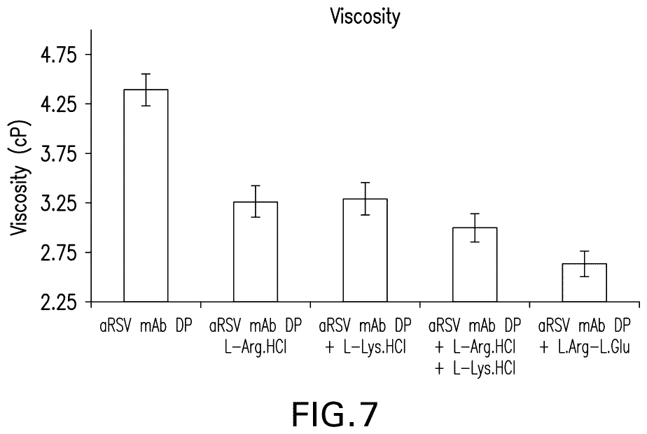

As shown in Table 5 and FIG. 7, all four sets of excipients decrease the viscosity of the anti-RSV antibody DP.

TABLE 5

| Sample Number | Samples | Viscosity (cP) |
|---|---|---|
| S1 | Anti-RSV Antibody | 4.3940 ± 0.1265 |
| S2 | Anti-RSV Antibody + L-Arg•HCl | 3.2658 ± 0.1585 |
| S3 | Anti-RSV Antibody + L-Lys•HCl | 3.2916 ± 0.1633 |
| S4 | Anti-RSV Antibody + (L-Arg•HCl + | 2.9982 ± 0.1435 |
| | L-Lys•HCl) | |
| S5 | Anti-RSV Antibody + (L-Arg + L-Glu) | 2.6344 ± 0.1288 |

### Example 5

### Addition of Chelator to Anti-RSV Antibody Formulation to Decrease Aggregation

This study compared the stability of an anti-RSV antibody in 10 mM histidine (pH=6), 0.02% PS80, 7% sucrose in the presence or absence of 50 μM DTPA (Diethylenetriamine pentaacetate).

To evaluate the stability of the formulations, two formulations were filled into vials and staged on stability at 5° C. (ambient humidity), 25° C. (60% relative humidity), and 40° C. (75% relative humidity) for eight weeks protected from light. The two formulations are shown in Table 6.

TABLE 6

| Formulation | | | | | |
|---|---|---|---|---|---|
| Number | Description | | | | |
| 1 | Anti-RSV | 10 mM | 7% | 0.02% PS80 | N/A |
| | antibody | Histidine | Sucrose | (w/v) | |
| | (100 mg/mL) | (pH = 6) | (w/v) | | |
| 2 | Anti-RSV | 10 mM | 7% | 0.02% PS80 | 50 μM |
| | antibody | Histidine | Sucrose | (w/v) | DTPA |
| | (100 mg/mL) | (pH = 6) | (w/v) | | |

Samples were assessed by size exclusion chromatography (SEC) for purity in which the percentage of monomer was determined, as well as the percentages of high molecular weight species (HMW) and late eluting peaks (LMW species). Ultra Performance—Size Exclusion Chromatography (UP-SEC) was performed by diluting the samples to 1.0 mg/mL in mobile phase (100 mM sodium phosphate and 100 mM sodium chloride, pH 7.0); flow rate of 0.5 mL/min. The diluted samples were injected (5 μL) into a UPLC equipped with a Waters BEH200 column and a UV detector. Proteins in the sample were separated by size and detected by UV absorption at 214 nm.

The UPSEC data to evaluate the levels of High Molecular Weight Species (HMW or aggregates), 00 monomer and LMW (Low Molecular Weight species) is shown in Table 7. [T0=Time 0; 4W=4 Weeks; and 8W=8 Weeks]

TABLE 7

| | Peak | | |
|---|---|---|---|
| Sample Name | HMW | Monomer | LMW |
| Anti-RSV Antibody DP control T05° C. | 1.77 | 98.1 | 0.09 |
| Anti-RSV Antibody DP control T0 4W5° C. | 1.88 | 98.0 | 0.11 |
| Anti-RSV Antibody DP control T0 4W25° C. | 2.86 | 96.9 | 0.21 |
| Anti-RSV Antibody DP control T0 4W40° C. | 4.05 | 93.2 | 2.77 |
| Anti-RSV Antibody DP control T0 8W5° C. | 2.00 | 97.9 | 0.13 |
| Anti-RSV Antibody DP control T0 8W25° C. | 3.13 | 96.6 | 0.28 |
| Anti-RSV Antibody DP control T0 8W40° C. | 4.83 | 91.1 | 4.05 |
| Anti-RSV Antibody DP + DTPA T05° C. | 1.72 | 98.2 | 0.12 |
| Anti-RSV Antibody DP + DTPA 4W5° C. | 1.81 | 98.1 | 0.11 |
| Anti-RSV Antibody DP + DTPA 4W25° C. | 2.53 | 97.3 | 0.19 |
| Anti-RSV Antibody DP + DTPA 4W40° C. | 3.24 | 94.7 | 2.02 |
| Anti-RSV Antibody DP + DTPA 8W5° C. | 1.87 | 98.0 | 0.12 |
| Anti-RSV Antibody DP + DTPA 8W25° C. | 2.68 | 97.1 | 0.21 |
| Anti-RSV Antibody DP + DTPA 8W40° C. | 3.50 | 93.8 | 2.68 |

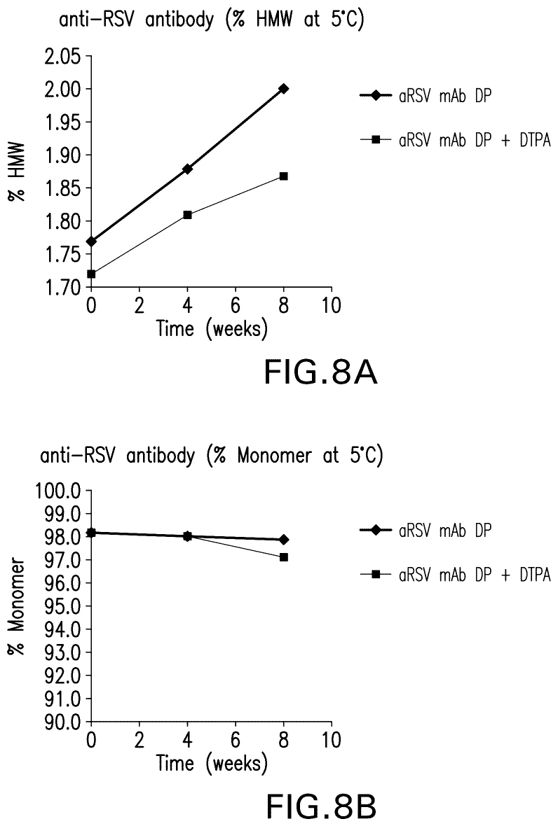

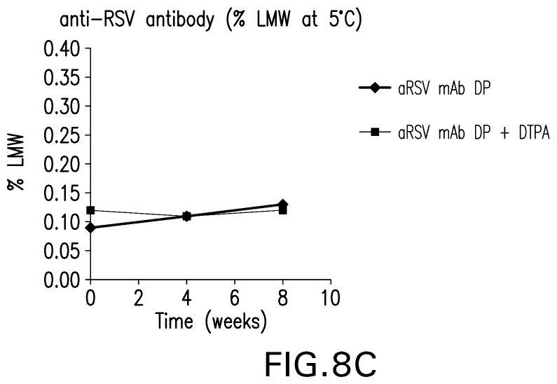

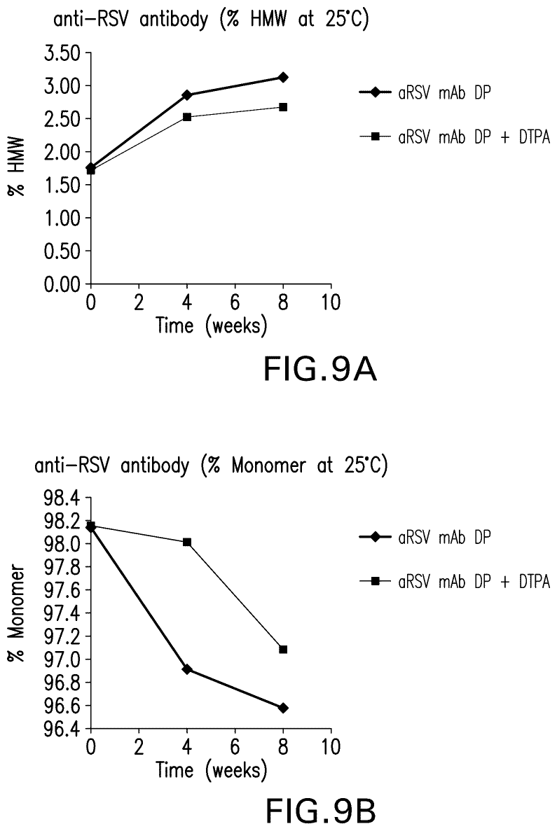

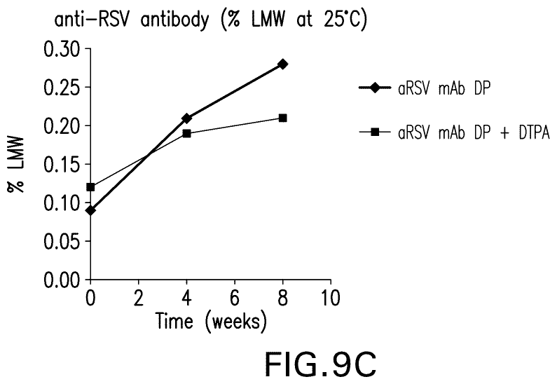

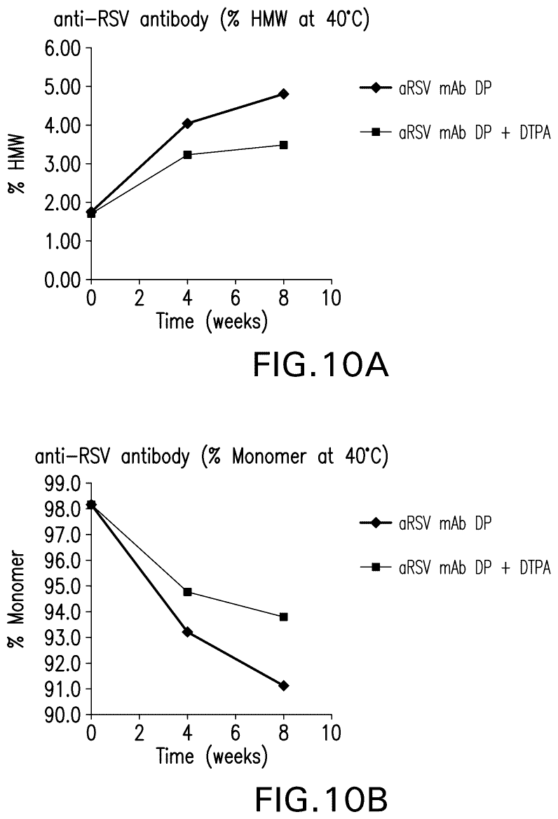

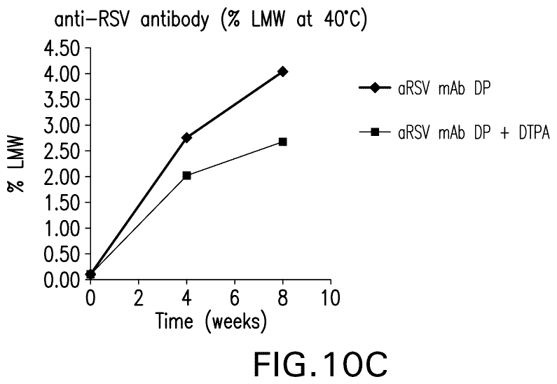

As shown in FIG. 8, FIG. 9 and FIG. 10, UP-SEC analysis of the samples to determine the percentage of HMW and percentage of monomer indicated that at 5° C., 25° C. and 40° C., both formulations showed a trend towards an increase in % HMW peak and % LMW peak (and a consequent decrease in 00 monomer peak) up to the 8-week time point. At 25° C., both formulations showed similar trends, but smaller changes, as compared to 40° C. At 5° C., no substantial changes were observed.

As seen in FIGS. 9 and 10, formulation 1 showed a greater increase in % HMW and % LMW as compared to formulation 2. Additionally, formulation 1 showed a greater decrease of 0 monomer as compared to formulation 2. Thus, DTPA (formulation 2) decreases the % HMW and % LMW and thus improves the aggregation of the anti-RSV antibody DP.

### Example 6

### Addition of Chelator to Anti-RSV Antibody Formulation to Improve Chemical Stability of the Anti-RSV Antibody

This study compares the chemical stability of an anti-RSV antibody in 10 mM histidine (pH=6), 0.02% PS80, 7% sucrose in the presence or absence of 50 μM DTPA.

To evaluate the chemical stability of the formulations, two formulations were filled into vials and staged on stability at 5° C. (ambient humidity), 25° C. (60% relative humidity), and 40° C. (75% relative humidity) for eight weeks protected from light. The two formulations are as provided in Example 5.

Ion exchange chromatography was performed to evaluate the chemical stability and to monitor the change in the charge variant profile over time. An ion exchange HPLC method was performed using a Dionex ProPac WCX-10 column and a UV detector at 280 nm. Samples were diluted in purified water, and 80 μg were injected for analysis. The mobile phase used for the IEX analysis of the thermal stability samples was a gradient of the following mobile phases (mobile phase A:20 mM MOPS, pH 7.2; mobile phase B: 50 mM sodium phosphate, 60 mM sodium chloride pH 8.0). The assay was performed using a mobile phase gradient from 20 mM MOPS, pH 7.2 to 50 mM sodium phosphate, 60 mM NaCl, pH 8.0. UV detection was performed at 280 nm.

The HP-IEX data to evaluate the levels of Acidic Variants, % Main Peak and % Basic Variants is shown in Table 8. [T0=Time 0; 4W=4 Weeks; and 8W=8 Weeks]

TABLE 8

| | Reportable % Peak Areas | | |
|---|---|---|---|
| | Acidic | Total | Basic |
| Sample Name | Variants | Main | Variants |
| Anti-RSV Antibody DP control T05° C. | 25.18 | 58.1 | 16.68 |
| Anti-RSV Antibody DP control T05° C. | 24.97 | 58.9 | 16.10 |
| 4W5° C. | | | |
| Anti-RSV Antibody DP control T05° C. | 28.41 | 54.1 | 17.47 |
| 4W25° C. | | | |
| Anti-RSV Antibody DP control T05° C. | 44.06 | 37.5 | 18.41 |
| 4W40° C. | | | |
| Anti-RSV Antibody DP control T05° C. | 24.57 | 59.0 | 16.38 |
| 8W5° C. | | | |
| Anti-RSV Antibody DP control T05° C. | 30.62 | 52.0 | 17.38 |
| 8W25° C. | | | |
| Anti-RSV Antibody DP control T05° C. | 53.49 | 28.7 | 17.82 |
| 8W40° C. | | | |
| Anti-RSV Antibody DP + DTPA T05° C. | 22.96 | 61.5 | 15.54 |
| Anti-RSV Antibody DP + DTPA 4W5° C. | 23.00 | 61.8 | 15.19 |
| Anti-RSV Antibody DP + DTPA 4W25° C. | 24.32 | 58.4 | 17.26 |
| Anti-RSV Antibody DP + DTPA 4W40° C. | 35.11 | 45.4 | 19.47 |
| Anti-RSV Antibody DP + DTPA 8W5° C. | 22.79 | 61.0 | 16.25 |
| Anti-RSV Antibody DP + DTPA 8W25° C. | 25.25 | 56.8 | 17.95 |
| Anti-RSV Antibody DP + DTPA 8W40° C. | 40.90 | 39.2 | 19.86 |

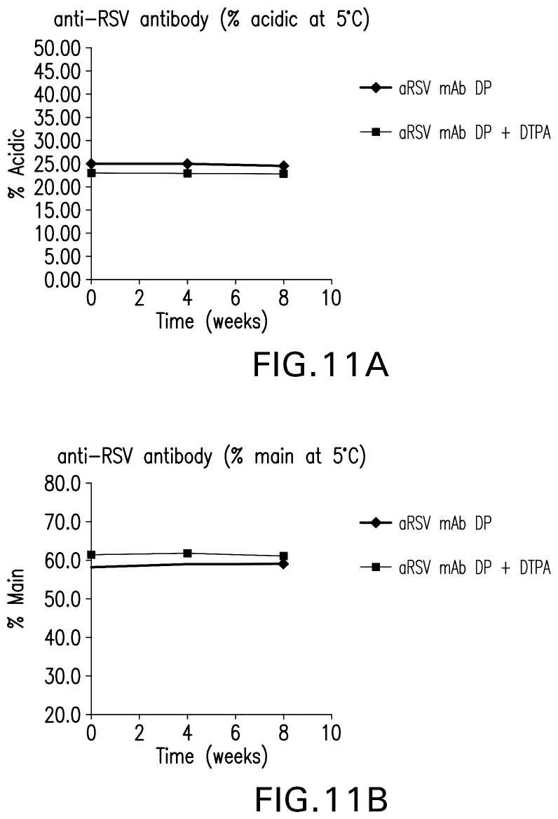

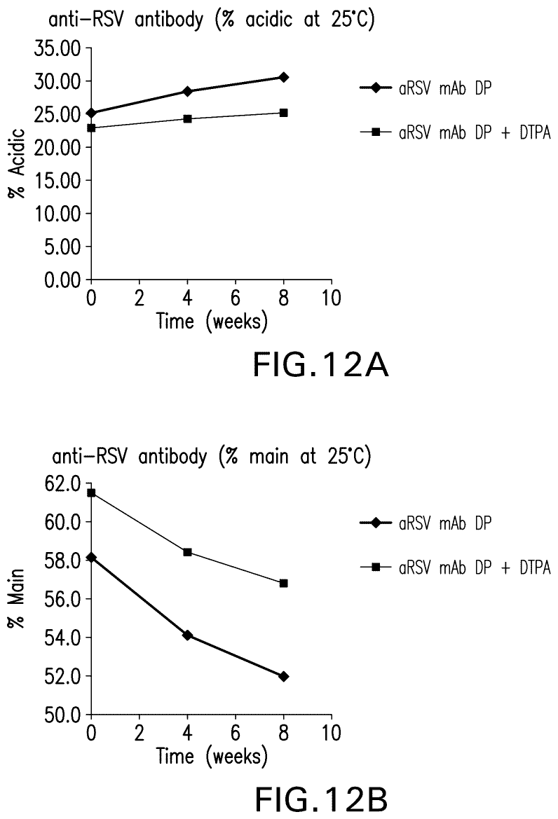

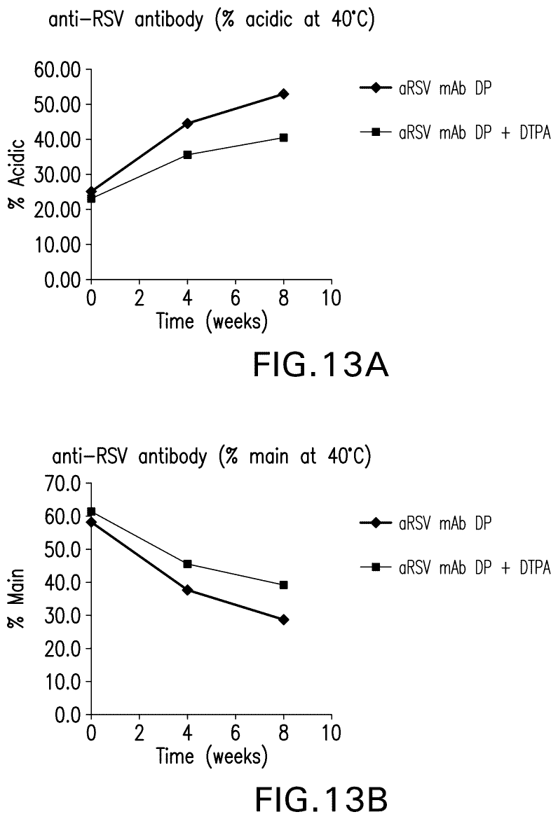

As shown in FIG. 11, FIG. 12 and FIG. 13, HIP-IEX analysis of the samples to determine chemical stability indicated that at 5° C., 25° C. and 40° C., both formulations showed a trend towards an increase in 00 acidic peak and a consequent decrease in 0% monomer peak) up to the 8-week time point. At 25° C., both formulations showed similar trends, but smaller changes, as compared to 40° C. At 5° C., no substantial changes were observed.

As seen in FIGS. 12 and 13, formulation 1 showed a greater increase in % acidic peak as compared to formulation 2. Additionally, formulation 1 showed a greater decrease of % main peak as compared to formulation 2. Thus, DTPA (formulation 2) decreased the % acidic peak and thus improves the chemical stability of an anti-RSV antibody DP.

### Example 7

### Addition of Chelator to Anti-RSV Antibody Formulation to Decrease PS80 Degradation

This study compared the chemical stability of PS80 in an anti-RSV antibody in 10 mM histidine (pH=6), 0.02% PS80, 7% sucrose in the presence or absence of 50 μM DTPA. Degradation of PS80 is commonly seen upon long-term storage of mAbs and the experiment was performed to determine if DTPA is compatible with PS80.

To evaluate the chemical stability of PS80 in two formulations, the two formulations were filled into vials and staged on stability at 5° C. (ambient humidity), 25° C. (60% relative humidity), and 40° C. (75% relative humidity) for eight weeks protected from light. The two formulations are as provided in Example 5.

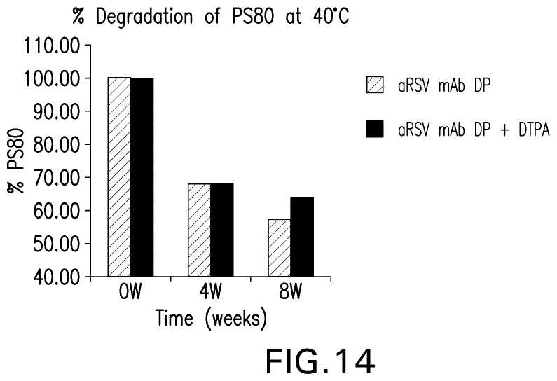

The results in Table 9 show similar degradation profiles of PS80 for both formulations. FIG. 14 shows the % degradation of PS80 at 40° C. The % degradation was slightly less for formulation 2 (in the presence of DTPA). Thus, DTPA might protect against PS80 degradation and these two excipients (DTPA and PS80) are mutually compatible. [T0=Time 0; 4W=4 Weeks; and 8W=8 Weeks]

TABLE 9

| | PS80 |
|---|---|
| Sample Name | concentration |
| Anti-RSV Antibody DP control T05° C. | 0.28 |
| Anti-RSV Antibody DP control T05° C. 4W5° C. | 0.27 |
| Anti-RSV Antibody DP control T05° C. 4W25° C. | 0.21 |
| Anti-RSV Antibody DP control T05° C. 4W40° C. | 0.19 |
| Anti-RSV Antibody DP control T05° C. 8W5° C. | 0.25 |
| Anti-RSV Antibody DP control T05° C. 8W25° C. | 0.18 |
| Anti-RSV Antibody DP control° T05° C. 8W40° C. | 0.16 |
| Anti-RSV Antibody DP + DTPA T05° C. | 0.25 |
| Anti-RSV Antibody DP + DTPA 4W5° C. | 0.23 |
| Anti-RSV Antibody DP + DTPA 4W25° C. | 0.19 |
| Anti-RSV Antibody DP + DTPA 4W40° C. | 0.17 |
| Anti-RSV Antibody DP + DTPA 8W5° C. | 0.23 |
| Anti-RSV Antibody DP + DTPA 8W25° C. | 0.16 |
| Anti-RSV Antibody DP + DTPA 8W40° C. | 0.16 |

### Example 8

### Increasing Excipient Concentrations to Decrease Viscosity and Maintain Stability

To assess the effect of increased concentrations of excipients to decrease viscosity, anti-RSV antibody was formulated at two different concentrations (around 200 mg/ml and 154 mg/mL) into four different formulations: a first formulation of 10 mM histidine, 700 sucrose and 0.0200 PS80, pH6, and three formulations adding an additional high concentration of excipient: 70 mM Histidine (for a total concentration of 80 mM histidine), 70 mM Lysine, or 70 mM Arg.

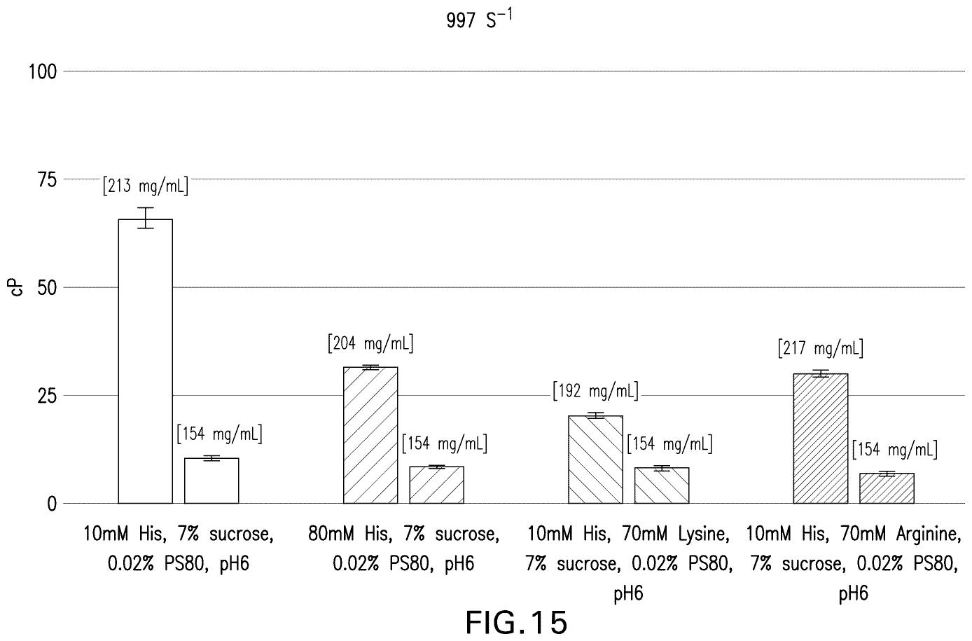

To measure the viscosity of the four formulations, the samples were loaded into a 500 μl syringe and viscosity was measured (four times for each sample) at room temperature with an MVROC Viscometer. VROC sensors detected viscosity by measuring the pressure drop as samples flow through the sensor's flow channel at designated positions from the inlet. Viscosity was measured as a function of shear rate (measured in reciprocal seconds [S⁻¹]). The shear rate used was 997 S-1. FIG. 15 shows a bar graph comparing the four different formulations of anti-RSV antibody at their respective concentrations (around 200 mg/ml and at 154 mg/mL).

Compared to a formulation of 10 mM histidine, 7% sucrose and 0.02% PS80, pH6, the addition of higher concentrations of histidine, lysine, and arginine showed a substantial decrease in viscosity. As shown in FIG. 15, the four formulations did not show a significantly different viscosity at an anti-RSV antibody concentration of 154 mg/mL. However, at the higher anti-RSV antibody concentrations of around 200 mg/mL, the formulations using additional 70 mM histidine, 70 mM lysine, or 70 mM arginine showed a significant decrease in mean viscosity compared with the first formulation. Anti-RSV antibody at 213 mg/mL in the formulation of 10 mM histidine, 7% sucrose and 0.02% PS80, pH6 has a mean viscosity of 64.97 centipoise (cP). Using additional excipient of 70 mM histidine (total concentration of 80 mM histidine) reduced the mean viscosity of 204 mg/mL anti-RSV antibody formulation to 30.85 cP. Using 70 mM lysine as an excipient reduced the mean viscosity of 192 mg/mL anti-RSV antibody formulation to 19.81 cP. Using 70 mM arginine as an excipient reduced the mean viscosity of 217 mg/mL anti-RSV antibody formulation to 29.27 cP.

The stability of each of the formulations was tracked for 3 months at 5° C., 25° C. and 40° C. Samples were assessed by size exclusion chromatography (SEC) for purity in which the percentage of monomer was determined, as well as the percentages of high molecular weight species (HMW) and late eluting peaks (LMW species). Ion exchange chromatography was performed as described in Example 6 to evaluate the chemical stability and to monitor the change in the charge variant profile over time. Values are listed in Table 10 below.

TABLE 10

| | | | 10 mM His, | 10 mM His, |
|---|---|---|---|---|
| | 10 mM His | 80 mM His | 70 mM Lys | 70 mM Arg |
| | (213 mg/mL) | (204 mg/mL) | (192 mg/mL) | (217 mg/mL) |
| Month 0, | 97.2/97.2/97.2 | 98.0/98.0/98.0 | 97.7/97.7/97.7 | 97.6/97.6/97.6 |
| Monomer % | | | | |
| (5° C./25° C./40° C.) | | | | |
| Month 0, | 2.4/2.4/2.4 | 1.6/1.6/1.6 | 1.8/1.8/1.8 | 1.9/1.9/1.9 |
| High Mol. Wt. % | | | | |
| (5° C./25° C./40° C.) | | | | |
| Month 0, | 0.4/0.4/0.4 | 0.4/0.4/0.4 | 0.5/0.5/0.5 | 0.5/0.5/0.5 |
| Low Mol. Wt. % | | | | |
| (5° C./25° C./40° C.) | | | | |
| Month 3, | 96.9/93.4/26.5 | 97.7/96.3/36.8 | 97.3/62.4/52.2 | 97.3/94.4/3.8 |
| Monomer % | | | | |
| (5° C./25° C./40° C.) | | | | |
| Month 3, | 2.4/4.0/44.2 | 1.6/2.4/40.3 | 1.9/34.6/37.2 | 1.9/2.5/65.4 |
| High Mol. Wt. % | | | | |
| (5° C./25° C./40° C.) | | | | |
| Month 3, | 0.7/2.6/29.3 | 0.7/1.3/22.9 | 0.8/3.1/10.6 | 0.8/3.1/30.9 |
| Low Mol. Wt. % | | | | |
| (5° C./25° C./40° C.) | | | | |

Compared to a formulation of 10 mM histidine, 700 sucrose and 0.0200 PS80, pH6, the addition of higher concentrations of histidine, lysine, and arginine did not lead to a significant increase of high molecular weight species or low molecular weight species, and did not lead to a significant decrease in the percentage monomer.

What is claimed is:

1. An anti-RSV antibody formulation comprising:
a) about 50 mg/mL to about 250 mg/mL of an anti-RSV antibody, or antigen-binding fragment thereof,
b) about 5 mM to about 20 mM histidine;
c) about 4% to about 8% (w/v) sucrose;
d) an excipient selected from the group consisting of
about 25 mM to about 75 mM of L-arginine or a pharmaceutically acceptable salt of L-arginine,
about 25 mM to about 75 mM of L-lysine or a pharmaceutically acceptable salt of L-lysine,
a mixture of about 25 mM to about 75 mM L-arginine or a pharmaceutically acceptable salt of L-arginine and about 25 mM to about 75 mM L-glutamate or a pharmaceutically acceptable salt of L-glutamate, and
a mixture of about 25 mM to about 75 mM of L-arginine or a pharmaceutically acceptable salt of L-arginine and about 25 mM to about 75 mM L-lysine or a pharmaceutically acceptable salt of L-lysine; and
e) about 0.01% to about 0.10% (w/v) polysorbate 80.

2. The anti-RSV antibody formulation of claim 1 comprising:
a) about 50 mg/ml to about 250 mg/ml of an anti-RSV antibody, or antigen-binding fragment thereof,
b) about 5 mM to about 20 mM histidine;
c) about 4% to about 8% (w/v) sucrose;
d) about 25 mM to about 75 mM of L-arginine or a pharmaceutically acceptable salt of L-arginine; and
e) about 0.01% to about 0.10% (w/v) polysorbate 80.

3. The anti-RSV antibody formulation of claim 1 comprising:
a) about 50 mg/ml to about 250 mg/ml of an anti-RSV antibody, or antigen-binding fragment thereof,
b) about 5 mM to about 20 mM histidine;
c) about 4% to about 8% (w/v) sucrose;
d) about 25 mM to about 75 mM of L-lysine or a pharmaceutically acceptable salt of L-lysine; and
e) about 0.01% to about 0.10% (w/v) polysorbate 80.

4. The anti-RSV antibody formulation of claim 1 comprising:
a) about 50 mg/ml to about 250 mg/ml of an anti-RSV antibody, or antigen-binding fragment thereof,
b) about 5 mM to about 20 mM histidine;
c) about 4% to about 8% (w/v) sucrose;
d) a mixture of about 25 mM to about 75 mM L-arginine or a pharmaceutically acceptable salt of L-arginine and about 25 mM to about 75 mM L-glutamate or a pharmaceutically acceptable salt of L-glutamate; and
e) about 0.01% to about 0.10% (w/v) polysorbate 80.

5. The anti-RSV antibody formulation of claim 1 comprising:
a) about 50 mg/ml to about 250 mg/ml of an anti-RSV antibody, or antigen-binding fragment thereof,
b) about 5 mM to about 20 mM histidine;
c) about 4% to about 8% (w/v) sucrose;
d) a mixture of about 25 mM to about 75 mM of L-arginine or a pharmaceutically acceptable salt of L-arginine and about 25 mM to about 75 mM L-lysine or a pharmaceutically acceptable salt of L-lysine; and
e) about 0.01% to about 0.10% (w/v) polysorbate 80.

6. The anti-RSV antibody formulation of claim 1, comprising about 125 mg/mL to about 175 mg/mL of the anti-RSV antibody, or antigen-binding fragment thereof.

7. The anti-RSV antibody formulation of claim 1, comprising about 150 mg/ml of the anti-RSV antibody, or antigen-binding fragment thereof.

8. The anti-RSV antibody formulation of claim 1, comprising about 150 mg/mL to about 200 mg/mL of the anti-RSV antibody, or antigen-binding fragment thereof.

9. The anti-RSV antibody formulation of claim 1, comprising about 175 mg/ml of the anti-RSV antibody, or antigen-binding fragment thereof.

10. The anti-RSV antibody formulation of claim 1, comprising about 175 mg/mL to about 225 mg/mL of the anti-RSV antibody, or antigen-binding fragment thereof.

11. The anti-RSV antibody formulation of claim 1, comprising about 200 mg/ml of the anti-RSV antibody, or antigen-binding fragment thereof.

12. The anti-RSV antibody formulation of claim 1, further comprising about 25 to about 75 μM diethylenetriamine pentaacetate (DTPA).

13. The anti-RSV antibody formulation of claim 1, further comprising about 1 mM to about 20 mM L-methionine.

14. The anti-RSV antibody formulation of claim 1, wherein the pH is 5.5 to 6.5.

15. The anti-RSV antibody formulation of claim 1, wherein the pH is 6.0.

16. The anti-RSV antibody formulation of claim 1, wherein the anti-RSV antibody comprises CDRs having the amino acid sequences of SEQ ID NOs: 1-6.

17. The anti-RSV antibody formulation of claim 1, wherein the anti-RSV antibody comprises a heavy chain variable region having the amino acid sequence of SEQ ID NO: 7 and a light chain variable region having the amino acid sequence of SEQ ID NO: 8.

18. The anti-RSV antibody formulation of claim 1, wherein the anti-RSV antibody comprises a heavy chain immunoglobulin consisting of the amino acid sequence set forth in SEQ ID NO: 9 and a light chain immunoglobulin consisting of the amino acid sequence set forth in SEQ ID NO: 10.

19. A method of treating or preventing RSV infection in a human patient in need thereof comprising administering an effective amount of the anti-RSV antibody formulation of claim 1 to the patient.

20. The method of claim 19, wherein the effective amount of the anti-RSV antibody formulation comprises a fixed dose of anti-RSV antibody or antigen-binding fragment thereof of about 10 mg to about 150 mg.

21. The method of claim 19, wherein the effective amount of the anti-RSV antibody formulation comprises a fixed dose of anti-RSV antibody or antigen-binding fragment thereof between about 25 mg and about 125 mg.

22. The method of claim 19, wherein the effective amount of the anti-RSV antibody formulation comprises a fixed dose of anti-RSV antibody or antigen-binding fragment thereof between about 50 mg and about 100 mg.

23. The method of claim 19, wherein the effective amount of the anti-RSV antibody formulation comprises a fixed dose of anti-RSV antibody or antigen-binding fragment thereof of about 100 mg.

24. The method of claim 19, wherein the anti-RSV antibody formulation is administered by intramuscular administration.
